(19) United States
(12) Patent Application Publication
(10) Pub. No.: US 2023/0295552 A1
(43) Pub. Date: Sep. 21, 2023

(54) LASER-ACTUATED SUPERCRITICAL INJECTOR

(71) Applicant: The Regents of the University of California, Oakland, CA (US)

(72) Inventors: Pei-Yu E. Chiou, Los Angeles, CA (US); Tianxing Man, Los Angeles, CA (US)

(73) Assignee: The Regents of the University of California, Oakland, CA (US)

(21) Appl. No.: 18/019,080

(22) PCT Filed: Aug. 3, 2021
(86) PCT No.: PCT/US21/44339

§ 371 Date: Jan. 31, 2023

Related U.S. Application Data
(60) Provisional application No. 62/706,152, filed on Aug. 3, 2020.

(51) Int. Cl. C12M 1/42; B23K 26/0622; B23K 26/06; C12N 9/22; C12N 15/87

(52) U.S. Cl. CPC C12M 35/02; B23K 26/0622; B23K 26/0648; C12N 9/22; C12N 15/87

(57) ABSTRACT

In various embodiments, a Laser-actuated Supercritical Injector (LASI) is provided. This device provides high-speed fluidic jet injection into biological samples, such as cells, organs, and tissues (including skin). In certain embodiments the LASI devices exploit high-speed fluidic jets that are pushed by rapid bubble expansion in a fluid. The bubbles are formed when liquid confined in microcavities or holes are heated up to above the supercritical temperature of the fluid. This leads to the formation of a short but ultra-high vapor pressure (supercritical) fluid that ejects the fluid (and any cargo contained therein) out through microchannels. This jet penetrates a cell, organ or tissue juxtaposed to a surface containing the microchannels and the jet provide sufficient force to penetrate into the cell, tissue, or organ leading to effective deliver of a cargo.

### CROSS-REFERENCE TO RELATED APPLICATIONS

This application claims priority to and benefit of U.S. Ser. No. 62/706,152, filed on Aug. 3, 2020, which is incorporated herein by reference in its entirety for all purposes.

### STATEMENT OF GOVERNMENTAL SUPPORT

This invention was made with government support under Grant Number FA9550-15-1-0406, awarded by the AFOSR. The government has certain rights in the invention.

### BACKGROUND

The intracellular delivery of nuclei acids, proteins and nano-devices are of great importance in the biomedical application, such as gene editing, cell-based therapies, and stem cell programming.^{[1]} Cargoes of interest range from small molecules of around 1 nm to large cellular or subcellular components of several microns.^{[2]} Delivery of large cargoes, such as organelles, subcellular components, and synthetic devices, has facilitated research in fields such as metabolic study, gene therapy, and intracellular environment probing.^{[3-6]}

Recent advances in CRISPR (clustered regularly interspaced short palindromic repeats) associated protein Cas9, for example, have achieved precise and targeted gene editing, while the delivery methods capable of transferring large chromosomes for insertion and correction are now in high demand.^{[7-8]}

Currently available methods mainly fall into two categories, with one utilizing various carriers to transport cargoes into cytosol through endosomal escape or membrane fusion, the other actively disrupting cell membrane for cargoes to migrate inside. Two major carriers in the first category are viral vectors and chemical vectors. Viral vectors take advantage of viral infection to enter the cellular cytosol and have been widely applied and proven effective in the delivery of a variety of cargoes. However, packing capacity and potential immunogenicity issues remain a major concern with respect to these delivery systems.^{[9-12]} Synthetic chemical, such as cationic lipid, polymers, and inorganic nanomaterials, have been utilized to package cargoes inside protect them from the internalization process. This packaging, however, may result in delayed drug release and low efficiency for hard-to-transfect cells.^{[13-15]}

Physical delivery methods, unlike carrier-based strategies, transfer cargoes into cellular cytosol by disrupting the cell membrane and increasing the membrane permeability, allowing more flexibility in cargo types. Physical delivery processes mainly undergo three phases: membrane disruption, cargo transport, and membrane recovery. The time window between membrane disruption and recovery is known to be only several seconds, limiting the time for cargoes to migrate into the cell.^{[1, 16]} For a large portion of physical delivery platforms, cargoes rely on diffusion depending on the concentration gradient between the two sides of the cell membrane, which can result in significantly slow migration speed and low delivery efficiency of large-sized cargoes.^{[17-19]} Thus, active cargo transport has been widely applied in platforms aiming for large cargo delivery.^{[, 20-24]}

Microinjection has dominated large cargo delivery for ages, with its capability to pierce through membrane and inject cargoes directly into the cytosol. However, microinjection is a low throughput transfection method.^{[5, 25, 26]} Ballistic injection directly delivers cargoes, precipitated on metallic micro and nanoparticles, into the cell cytoplasm or nucleus as a projectile ejected from a highly pressurized ballistic device. Ballistic injection, however, results in random distribution of injected material and excessive material injection.^{[23, 24, 27-29]}

Photothermal effects have been extensively applied in intracellular delivery field over the years utilizing specially designed metallic nanoparticles or nanostructures.^{[17, 20, 21, 30-34]} In systems utilizing photothermal effects, laser irradiation heats up s light-absorbed material to the critical temperature of the surrounding aqueous medium. Cavitation bubbles are generated at the interface between the light-absorbing material and the aqueous medium.^{[35, 36]} Based on the phase diagram of water, as an example, the initial pressure of the cavitation bubbles can be as high as 20 MPa, which results in explosive bubble expansion. These high-pressure bubbles have been utilized to open transient pores on an adjacent cell membrane, serving as the transfer channel for external cargoes.^{[17, 19, 32]} Our group has developed a high-throughput delivery platform using cavitation bubbles to create openings on adjacent cell membrane, followed by fluidic pumping to actively push cargoes through the opening.^{[20]} It achieved high-throughput delivery of micron-sized bacteria and mitochondria while maintaining high cell viability.

### SUMMARY

In various embodiments, a Laser-actuated Supercritical Injector (LASI), a device platform that allows high-speed fluidic jet injection into biological samples, such as cells, organs, and tissues (including skin) and methods of use thereof are provided. In certain embodiments the injector devices exploit high-speed fluidic jets that are pushed by bubble explosion in a short period of time. The bubbles are formed when liquid confined in microcavities or holes are heated up to above the supercritical temperature of the fluid. This leads to the formation of a short but ultra-high vapor pressure (e.g., on the order of tens or hundreds of MPa) (supercritical) fluid that ejects the fluid (and any cargo contained therein) out through microchannels. This jet penetrates a cell, organ or tissue juxtaposed to a surface containing the microchannels and the jet provide sufficient force to penetrate into the cell, tissue, or organ leading to effective deliver of a cargo.

Accordingly, various embodiments contemplated herein may include, but need not be limited to, one or more of the following:

Embodiment: 1: A laser-actuated supercritical injector (LASI) for delivery of a cargo into a cell or tissue, said injector comprising:
a substrate comprising a first layer, and optionally comprising a second layer, where said substrate defines an outer surface and where said substrate comprises a plurality of chambers disposed within the substrate where each chamber comprising said plurality of chambers is in fluid communication with one or a plurality of microchannels leading from each chamber to said outer surface of said substrate where the microchannel(s) opens to the outer surface of said substrate; and
a pulse laser configured to illuminate one or more of the chambers comprising said plurality of chambers, where said laser is configured to heat the walls of the illuminated chamber(s) and a fluid contained with the illuminated chamber(s) to transform said fluid into a supercritical fluid that ejects out to the surface of said substrate through the microchannel(s) opening into the illuminated chamber(s).

Embodiment 2: The laser-actuated supercritical injector of embodiment 1, wherein said substrate comprises a material that permits transmission of illumination from said laser to said plurality of chambers to permit heating of the walls of said chambers.

Embodiment 3: The laser-actuated supercritical injector of embodiment 2, wherein said substrate comprises a material that provides less than 10% attenuation, or less than 20% attenuation, or less than 30% attenuation, or less than 40% attenuation, or less than 50% attenuation, or less than 60% attenuation, or less than 70% attenuation, or less than 80% attenuation, or less than 90% attenuation, or less than 95% attenuation in said substrate at a depth of 500 μm.

Embodiment 4: The laser-actuated supercritical injector according of embodiment 1, wherein said substrate comprises silicon.

Embodiment 5: The laser-actuated supercritical injector according to any one of embodiments 1-4, wherein said substrate comprises a doped region.

Embodiment 6: The laser-actuated supercritical injector of embodiment 5, wherein said substrate comprises a lightly doped silicon substrate.

Embodiment 7: The laser-actuated supercritical injector according to any one of embodiments 5-6, wherein said doped silicon substrate comprises N doped silicon.

Embodiment 8: The laser-actuated supercritical injector according to any one of embodiments 5-6, wherein said doped silicon substrate comprises P doped silicon.

Embodiment 9: The laser-actuated supercritical injector according to any one of embodiments 1-8, wherein said substrate is doped at a level ranging from about 10¹³ ions/cm³ up to about 10²⁰ ions/cm³.

Embodiment 10: The laser-actuated supercritical injector according to any one of embodiments 1-9, wherein each chamber comprising said plurality of chambers is in fluid communication with a single microchannel leading from said chamber to the surface of said substrate.

Embodiment 11: The laser-actuated supercritical injector according to any one of embodiments 1-9, wherein said substrate comprises said second layer where said second layer and at least a portion of said microchannels are disposed in said second layer.

Embodiment 12: The laser-actuated supercritical injector of embodiment 11, wherein said second layer comprise a material selected from the group consisting of an oxide, a nitride, or a polymer.

Embodiment 13: The laser-actuated supercritical injector of embodiment 12, wherein said second layer comprises an oxide.

Embodiment 14: The laser-actuated supercritical injector of embodiment 13, wherein said oxide comprises SiO₂.

Embodiment 15: The laser-actuated supercritical injector according to any one of embodiments 1-14, wherein each chamber comprising said plurality of chambers is in fluid communication with one microchannel

Embodiment 16: The laser-actuated supercritical injector according to any one of embodiments 1-14, wherein each chamber comprising said plurality of chambers is in fluid communication with a plurality of microchannels.

Embodiment 17: The laser-actuated supercritical injector of embodiment 15, wherein each chamber comprising said plurality of chambers is in fluid communication with 2, 3, 4, 5, 6, 7, 8, 9, or 10 microchannels.

Embodiment 18: The laser-actuated supercritical injector according to any one of embodiments 1-17, wherein said plurality of chambers are disposed in a single depth (level) in said substrate.

Embodiment 19: The laser-actuated supercritical injector according to any one of embodiments 1-17, wherein said plurality of chambers are disposed in two or more depths (levels) in said substrate.

Embodiment 20: A laser-actuated supercritical injector (LASI) for delivery of a cargo into a cell or tissue, said injector comprising:
a substrate comprising a first layer, and optionally comprising a second layer, where said substrate defines an outer surface and where said substrate comprises a plurality of chambers disposed within the substrate where each chamber comprising said plurality of chambers is in fluid communication with one or a plurality of microchannels leading from each chamber to said outer surface of said substrate where the microchannel(s) open to the outer surface of said substrate, and where each chamber comprising said plurality of chambers comprises a doped region and/or a metal region that can survive heating to a temperature sufficient to transform a fluid within said chamber to a supercritical fluid when irradiated by a pulse laser; and
a pulse laser configured to illuminate one or more of the chambers comprising said plurality of chambers, where said laser is configured to heat said metal region(s) in the illuminated chamber(s) and a fluid contained with the illuminated chamber(s) to transform said fluid into a supercritical fluid that ejects out to the surface of said substrate through the microchannel(s) opening into the illuminated chamber(s).

Embodiment 21: The laser-actuated supercritical injector of embodiment 20, wherein said substrate comprises a material that permits transmission of illumination from said laser to said plurality of chambers to permit heating of the walls of said chambers.

Embodiment 22: The laser-actuated supercritical injector of embodiment 21, wherein said substrate comprises a material that provides less than 10% attenuation, or less than 20% attenuation, or less than 30% attenuation, or less than 40% attenuation, or less than 50% attenuation, or less than 60% attenuation, or less than 70% attenuation, or less than 80% attenuation, or less than 90% attenuation, or less than 95% attenuation in said substrate at a depth of 500 μm.

Embodiment 23: The laser-actuated supercritical injector of embodiment 20, wherein said substrate comprises silicon.

Embodiment 24: The laser-actuated supercritical injector according to any one of embodiments 20-23, wherein each chamber comprising said plurality of chambers is in fluid communication with a single microchannel leading from said chamber to the surface of said substrate.

Embodiment 25: The laser-actuated supercritical injector according to any one of embodiments 20-23, wherein each chamber comprising said plurality of chambers is in fluid communication with a plurality of microchannels leading from said chamber to the surface of said substrate.

Embodiment 26: The laser-actuated supercritical injector of embodiment 25, wherein each chamber comprising said plurality of chambers is in fluid communication with 2, 3, 4, 5, 6, 7, 8, 9, or 10 microchannels.

Embodiment 27: The laser-actuated supercritical injector according to any one of embodiments 20-26, wherein said plurality of chambers are disposed in a single depth (level) in said substrate.

Embodiment 28: The laser-actuated supercritical injector according to any one of embodiments 20-26, wherein said plurality of chambers are disposed in two or more depths (levels) in said substrate.

Embodiment 29: The laser-actuated supercritical injector according to any one of embodiments 20-28, wherein said substrate comprises said second layer where said second layer and at least a portion of said microchannels are disposed in said second layer.

Embodiment 30: The laser-actuated supercritical injector of embodiment 29, wherein said second layer comprise a material selected from the group consisting of an oxide, a nitride, or a polymer.

Embodiment 31: The laser-actuated supercritical injector of embodiment 30, wherein said second layer comprises an oxide.

Embodiment 32: The laser-actuated supercritical injector of embodiment 31, wherein said oxide comprises SiO₂.

Embodiment 33: The laser-actuated supercritical injector according to any one of embodiments 20-32, wherein each chamber comprising said plurality of chambers comprises a doped region.

Embodiment 34: The laser-actuated supercritical injector of embodiment 33, wherein each chamber comprising said plurality of chambers comprises a heavily doped region.

Embodiment 35: The laser-actuated supercritical injector according to any one of embodiments 33-34, wherein each chamber comprising said plurality of chambers comprises a P doped region.

Embodiment 36: The laser-actuated supercritical injector according to any one of embodiments 33-34, wherein each chamber comprising said plurality of chambers comprises an N doped region.

Embodiment 37: The laser-actuated supercritical injector according to any one of embodiments 20-32, wherein each chamber comprising said plurality of chambers comprises a metal region.

Embodiment 38: The laser-actuated supercritical injector of embodiment 37, wherein said metal region comprises a metal selected from the group consisting of gold, titanium (Ti), TiN, TiCn, TiAlN, and tungsten (W).

Embodiment 39: The laser-actuated supercritical injector of embodiment 38, said metal comprises titanium.

Embodiment 40: The laser-actuated supercritical injector according to any one of embodiments 20-39, wherein said metal region comprises a metal disk disposed within and at a wall of said chamber.

Embodiment 41: The laser-actuated supercritical injector according to any one of embodiments 20-39, wherein said metal region comprises a metal film deposited on the wall of said chamber.

Embodiment 42: The laser-actuated supercritical injector according to any one of embodiments 40-41, wherein said metal disk or metal film ranges from about 1 μm up to about 30 μm in average diameter.

Embodiment 43: The laser-actuated supercritical injector according to any one of embodiments 40-42, wherein said metal disk or metal film comprising said metal region ranges from about 0.05 μm up to about 1 μm in thickness.

Embodiment 44: The laser-actuated supercritical injector according to any one of embodiments 1-43, wherein the chambers comprising said plurality of chambers are substantially hemispheric.

Embodiment 45: The laser-actuated supercritical injector according to any one of embodiments 1-43, wherein the chambers comprising said plurality of chambers are substantially cylindrical, or substantially teardrop shaped, or substantially pyramidal shaped, or substantially conical shaped, or substantially triangular shaped.

Embodiment 46: The laser-actuated supercritical injector according to any one of embodiments 1-45, wherein the average volume of said chambers ranges from about 1 fL up to about 100 pL.

Embodiment 47: The laser-actuated supercritical injector of embodiment 46, wherein the average volume of said chambers is about 10 pL.

Embodiment 48: The laser-actuated supercritical injector according to any one of embodiments 1-47, wherein the average maximum diameter of said chambers ranges from about 1 μm up to about 200 μm.

Embodiment 49: The laser-actuated supercritical injector of embodiment 48, wherein the average maximum diameter of said chambers is about 80 μm.

Embodiment 50: The laser-actuated supercritical injector according to any one of embodiments 1-49, wherein said microchannels range in length from about 1 μm up to about 500 μm.

Embodiment 51: The laser-actuated supercritical injector of embodiment 50, wherein said microchannels have an average length of about 1 μm.

Embodiment 52: The laser-actuated supercritical injector according to any one of embodiments 1-51, wherein said microchannels range in average diameter from about 0.1 μm up to about 30 μm.

Embodiment 53: The laser-actuated supercritical injector of embodiment 52, wherein said microchannels have an average diameter of about 3 μm.

Embodiment 54: The laser-actuated supercritical injector according to any one of embodiments 1-53, wherein said substrate comprises at least about 50 microchannels, or at least about 100 microchannels, or at least about 500 microchannels, or at least about 1,000 microchannels, or at least about 2,500 microchannels, or at least about 5,000 microchannels, or at least about 7,500 microchannels, or at least about 10,000 microchannels up to about 4,000,000 microchannels, or up to about 3,000,000 microchannels, or up to about 2,000,000 microchannels, or up to about 1,000,000 microchannels, or up to about 500,000 microchannels, or up to about 250,000 microchannels, or up to about 100,000 microchannels, or up to about 50,000 microchannels.

Embodiment 55: The laser-actuated supercritical injector according to any one of embodiments 1-54, wherein said microchannels are present in said substrate at a density of at least about 50 microchannels/cm², or at least about 100 microchannels/cm², or at least about 500 microchannels/cm², or at least about 1,000 microchannels/cm², or at least about 2,500 microchannels/cm², or at least about 5,000 microchannels/cm², or at least about 7,500 microchannels/cm², or at least about 10,000 microchannels/cm² up to about 4,000,000 microchannels/cm², or up to about 3,000,000 microchannels/cm², or up to about 2,000,000 microchannels/cm², or up to about 1,000,000 microchannels/cm², or up to about 500,000 microchannels/cm², or up to about 250,000 microchannels/cm², or up to about 100,000 microchannels/cm², or up to about 50,000 microchannels/cm².

Embodiment 56: A laser-actuated supercritical injector (LASI) for delivery of a cargo into a cell, tissue, or organ said injector comprising: a substrate comprising a first layer, and optionally comprising a second layer, where said substrate defines an outer surface and comprises a plurality of microchannels, where each microchannel comprises a first end and a second end, where the first end opens to the outer surface of said substrate, and the second end of each microchannel is closed, terminating within said substrate; and a pulse laser configured to illuminate said substrate in a region comprising one or more of the microchannels comprising said plurality of microchannels, where said laser provides laser radiation having a power and wavelength sufficient to heat a fluid within the illuminated microchannels to transform said fluid into a supercritical fluid that ejects out through the illuminated microchannel(s).

Embodiment 57: The laser-actuated supercritical injector of embodiment 56, wherein said substrate comprises a material that permits transmission of illumination from said laser to said plurality of chambers to permit heating of the walls of said chambers.

Embodiment 58: The laser-actuated supercritical injector of embodiment 56, wherein said substrate comprises a material that provides less than 10% attenuation, or less than 20% attenuation, or less than 30% attenuation, or less than 40% attenuation, or less than 50% attenuation, or less than 60% attenuation, or less than 70% attenuation, or less than 80% attenuation, or less than 90% attenuation, or less than 95% attenuation in said substrate at a depth of 500 μm.

Embodiment 59: The laser-actuated supercritical injector according of embodiment 56, wherein said substrate comprises silicon.

Embodiment 60: The laser-actuated supercritical injector of embodiment 56-59, wherein said substrate comprises a doped substrate.

Embodiment 61: The laser-actuated supercritical injector of embodiment 60, wherein said substrate comprise a lightly doped substrate.

Embodiment 62: The laser-actuated supercritical injector of embodiment 61, wherein said lightly doped silicon substrate comprises an N doped substrate.

Embodiment 63: The laser-actuated supercritical injector of embodiment 61, wherein said lightly doped silicon substrate comprises a P doped substrate.

Embodiment 64: The laser-actuated supercritical injector according to any one of embodiments 56-63, wherein said substrate is doped at a level ranging from about 10¹⁴ to about 10¹⁵ ions/cm³.

Embodiment 65: The laser-actuated supercritical injector according to any one of embodiments 56-64, wherein said substrate comprises said second layer and at least a portion of said microchannels are disposed in said second layer.

Embodiment 66: The laser-actuated supercritical injector of embodiment 65, wherein said second layer comprises a material selected from the group consisting of an oxide, a nitride, or a polymer.

Embodiment 67: The laser-actuated supercritical injector of embodiment 66, wherein said second layer comprises an oxide.

Embodiment 68: The laser-actuated supercritical injector of embodiment 67, wherein said oxide comprises SiO₂.

Embodiment 69: The laser-actuated supercritical injector according to any one of embodiments 56-68, wherein said microchannels range in length from about said microchannels range in length from about 1 μm up to about 500 μm.

Embodiment 70: The laser-actuated supercritical injector of embodiment 69, wherein said microchannels have an average length of about 28 μm.

Embodiment 71: The laser-actuated supercritical injector of embodiment 69, wherein said microchannels have an average length of about 36 μm.

Embodiment 72: The laser-actuated supercritical injector according to any one of embodiments 56-70, wherein said microchannels range in average diameter from about 0.1 μm up to about 30 μm.

Embodiment 73: The laser-actuated supercritical injector of embodiment 72, wherein said microchannels have an average diameter of about 5 μm.

Embodiment 74: The laser-actuated supercritical injector of embodiment 72, wherein said microchannels have an average diameter of about 3 μm.

Embodiment 75: The laser-actuated supercritical injector according to any one of embodiments 56-74, wherein said substrate comprises at least about 50 microchannels, or at least about 100 microchannels, or at least about 500 microchannels, or at least about 1,000 microchannels, or at least about 2,500 microchannels, or at least about 5,000 microchannels, or at least about 7,500 microchannels, or at least about 10,000 microchannels up to about 4,000,000 microchannels, or up to about 3,000,000 microchannels, or up to about 2,000,000 microchannels, or up to about 1,000,000 microchannels, or up to about 500,000 microchannels, or up to about 250,000 microchannels, or up to about 100,000 microchannels, or up to about 50,000 microchannels.

Embodiment 76: The laser-actuated supercritical injector according to any one of embodiments 56-75, wherein said microchannels are present in said substrate at a density of at least about 50 microchannels/cm², or at least about 100 microchannels/cm², or at least about 500 microchannels/cm², or at least about 1,000 microchannels/cm², or at least about 2,500 microchannels/cm², or at least about 5,000 microchannels/cm², or at least about 7,500 microchannels/cm², or at least about 10,000 microchannels/cm² up to about 4,000,000 microchannels/cm^{2,} or up to about 3,000,000 microchannels/cm², or up to about 2,000,000 microchannels/cm^{2,} or up to about 1,000,000 microchannels/cm², or up to about 500,000 microchannels/cm², or up to about 250,000 microchannels/cm², or up to about 100,000 microchannels/cm², or up to about 50,000 microchannels/cm².

Embodiment 77: The laser-actuated supercritical injector according to any one of embodiments 1-76, wherein said pulse laser produces illumination at a wavelength ranging from about 380 nm up to about 2000 nm.

Embodiment 78: The laser-actuated supercritical injector of embodiment 77, wherein said pulse laser produces illumination at a wavelength ranging from about 380 nm up to about 1100 nm.

Embodiment 79: The laser-actuated supercritical injector according to any one of embodiments 1-78, wherein said pulse laser produces illumination at a power ranging from about 100 mJ/cm² up to about 1×10⁴ mJ/cm².

Embodiment 80: The laser-actuated supercritical injector according to any one of embodiments 1-79, wherein said pulse laser produces a green illumination.

Embodiment 81: The laser-actuated supercritical injector of embodiment 80, wherein said laser produces illumination at a wavelength of about 532 nm.

Embodiment 82: The laser-actuated supercritical injector according to any one of embodiments 80-81, wherein said laser produces illumination at a power of about 200 mJ/cm².

Embodiment 83: The laser-actuated supercritical injector according to any one of embodiments 1-79, wherein said pulse laser produces an infrared or a near infrared, or a far infrared illumination.

Embodiment 84: The laser-actuated supercritical injector of embodiment 83, wherein said laser produces illumination at a wavelength of about 1064 nm.

Embodiment 85: The laser-actuated supercritical injector according to any one of embodiments 83-84, wherein said laser produces illumination at a power of about 7.6×10³ mJ/cm².

Embodiment 86: The laser-actuated supercritical injector according to any one of embodiments 1-76, wherein said pulse laser is configured to illuminate a region of said substrate ranging from about 1 um² up to about 10 cm².

Embodiment 87: The laser-actuated supercritical injector of embodiment 86, wherein said pulse laser is configured to illuminate a region of said substrate about a 3 mm diameter.

Embodiment 88: The laser-actuated supercritical injector according to any one of embodiments 1-87, wherein said injector comprises a lens system, a mirror system, and/or a mask, and/or a positioning system to directing the laser radiation to a specific region of said substrate.

Embodiment 89: The laser-actuated supercritical injector according to any one of embodiments 1-88, wherein injector comprises an objective lens configured to focus optical energy onto said substrate.

Embodiment 90: The laser-actuated supercritical injector according to any one of embodiments 1-89, wherein said injector comprises a controller that adjusts at least one of the pattern of illumination by said laser, the timing of occurrence of light pulses emitted by the laser, the frequency of occurrence of pulses emitted by the laser, the wavelength of pulses emitted by the laser, the energy of pulses emitted by the laser, and the aiming or location of pulses emitted by the laser.

Embodiment 91: The laser-actuated supercritical injector according to any one of embodiments 1-90, wherein said injector comprises a controller that adjusts the x-y position of said substrate with respect to said laser.

Embodiment 92: The laser-actuated supercritical injector according to any one of embodiments 1-91, wherein said microchannels and/or said chambers when present, are loaded with a cargo.

Embodiment 93: The laser-actuated supercritical injector of embodiment 92, wherein said cargo is in solution or suspension in a aqueous solution.

Embodiment 94: The laser-actuated supercritical injector of embodiment 93, wherein said solution or suspension comprises a buffer.

Embodiment 95: The laser-actuated supercritical injector according to any one of embodiments 92-94, wherein said cargo comprises a moiety selected from the group consisting of a nucleic acid, a protein, a nucleic acid/protein complex, a carbohydrate, a small organic molecule, an organelle, a nanoparticle, a liposome, a natural chromosome or a natural chromosome fragment, a synthetic chromosome or synthetic chromosome fragment, an intracellular fungus, an intracellular protozoan, DNA and/or RNA packaged in a liposome or a lipid particle, and a vaccine comprising an antigen and an adjuvant.

Embodiment 96: The laser-actuated supercritical injector of embodiment 95, wherein said cargo comprises a cell nucleus, or a mitochondria.

Embodiment 97: The laser-actuated supercritical injector of embodiment 95, wherein said cargo comprises a nucleic acid encoding an enzyme.

Embodiment 98: The laser-actuated supercritical injector of embodiment 95, wherein said cargo comprises a moiety selected from the group consisting of a Zinc Finger Nuclease (ZFN), a nucleic acid encoding a ZFN, a Transcription Activator-Like Effector Nuclease (TALEN), a nucleic acid encoding a TALEN, a Clustered Regularly Interspaced

Short Palindromic Repeats (CRISPR)-associated protein, and a nucleic acid encoding a CRISPR protein.

Embodiment 99: The laser-actuated supercritical injector of embodiment 98, wherein said cargo comprises a nucleic acid encoding a CRISPR endonuclease protein and a guide RNA, or a CRISPR endonuclease protein and a guide RNA.

Embodiment 100: The laser-actuated supercritical injector of embodiment 99, wherein said CRISPR/Cas endonuclease protein comprises a class 2 CRISPR/Cas endonuclease and a guide RNA.

Embodiment 101: The laser-actuated supercritical injector of embodiment 100, wherein said class 2 CRISPR/Cas endonuclease is a type II CRISPR/Cas endonuclease.

Embodiment 102: The laser-actuated supercritical injector according to any one of embodiments 100-101, wherein the class 2 CRISPR/Cas endonuclease is a Cas9 polypeptide and the corresponding CRISPR/Cas guide RNA is a Cas9 guide RNA.

Embodiment 103: The laser-actuated supercritical injector of embodiment 102, wherein said Cas9 protein is selected from the group consisting of a Streptococcus pyogenes Cas9 protein (spCas9) or a functional portion thereof, a Staphylococcus aureus Cas9 protein (saCas9) or a functional portion thereof, a Streptococcus thermophilus Cas9 protein (stCas9) or a functional portion thereof, a Neisseria meningitides Cas9 protein (nmCas9) or a functional portion thereof, and a Treponema denticola Cas9 protein (tdCas9) or a functional portion thereof.

Embodiment 104: The laser-actuated supercritical injector according to any one of embodiments 100-101, wherein the class 2 CRISPR/Cas endonuclease is a type V or type VI CRISPR/Cas endonuclease.

Embodiment 105: The laser-actuated supercritical injector of embodiment 104, wherein the class 2 CRISPR/Cas endonuclease is selected from the group consisting of a Cpfl polypeptide or a functional portion thereof, a C2c1 polypeptide or a functional portion thereof, a C2c3 polypeptide or a functional portion thereof, and a C2c2 polypeptide or a functional portion thereof.

Embodiment 106: The laser-actuated supercritical injector according to any one of embodiments 1-105, wherein a cell tissue, or organ is juxtaposed to said surface of said substrate.

Embodiment 107: A method of introducing a cargo into a cell, tissue, or organ, said method comprising:
providing a laser-actuated supercritical injector according to any one of embodiments 1-91, wherein said microchannels and/or said chambers when present, are loaded with said cargo in a fluid;
juxtaposing said surface of said substrate to a cell, tissue, or organ; and
activating said pulse laser to illuminate at least a portion of said substrate and to heat said fluid and transform said fluid to a supercritical fluid that ejects out from said microchannels and injects into said cell, tissue, or organ.

Embodiment 108: The method of embodiment 106, wherein said cargo is in solution or suspension in a aqueous solution.

Embodiment 109: The method of embodiment 108, wherein said solution or suspension comprises a buffer.

Embodiment 110: The method according to any one of embodiments 106-109, wherein said cargo comprises a moiety selected from the group consisting of a nucleic acid, a protein, a nucleic acid/protein complex, a carbohydrate, a small organic molecule, an organelle, a nanoparticle, a liposome, a natural chromosome or a natural chromosome fragment, a synthetic chromosome or synthetic chromosome fragment, an intracellular fungus, an intracellular protozoan, DNA and/or RNA packaged in a liposome or a lipid particle, and a vaccine comprising an antigen and an adjuvant.

Embodiment 111: The method of embodiment 110, wherein said cargo comprises a cell nucleus, or a mitochondria.

Embodiment 112: The method of embodiment 110, wherein said cargo comprises a nucleic acid encoding an enzyme.

Embodiment 113: The method of embodiment 110, wherein said cargo comprises a moiety selected from the group consisting of a Zinc Finger Nuclease (ZFN), a nucleic acid encoding a ZFN, a Transcription Activator-Like Effector Nuclease (TALEN), a nucleic acid encoding a TALEN, a Clustered Regularly Interspaced Short Palindromic Repeats (CRISPR)-associated protein, and a nucleic acid encoding a CRISPR protein.

Embodiment 114: The method of embodiment 113, wherein said cargo comprises a nucleic acid encoding a CRISPR endonuclease protein and a guide RNA, or a CRISPR endonuclease protein and a guide RNA.

Embodiment 115: The method of embodiment 114, wherein said CRISPR/Cas endonuclease protein comprises a class 2 CRISPR/Cas endonuclease and a guide RNA.

Embodiment 116: The method of embodiment 115, wherein said class 2 CRISPR/Cas endonuclease is a type II CRISPR/Cas endonuclease.

Embodiment 117: The method according to any one of embodiments 115-116, wherein the class 2 CRISPR/Cas endonuclease is a Cas9 polypeptide and the corresponding CRISPR/Cas guide RNA is a Cas9 guide RNA.

Embodiment 118: The method of embodiment 117, wherein said Cas9 protein is selected from the group consisting of a Streptococcus pyogenes Cas9 protein (spCas9) or a functional portion thereof, a Staphylococcus aureus Cas9 protein (saCas9) or a functional portion thereof, a Streptococcus thermophilus Cas9 protein (stCas9) or a functional portion thereof, a Neisseria meningitides Cas9 protein (nmCas9) or a functional portion thereof, and a Treponema denticola Cas9 protein (tdCas9) or a functional portion thereof.

Embodiment 119: The method according to any one of embodiments 115-116, wherein the class 2 CRISPR/Cas endonuclease is a type V or type VI CRISPR/Cas endonuclease.

Embodiment 120: The method of embodiment 119, wherein the class 2 CRISPR/Cas endonuclease is selected from the group consisting of a Cpfl polypeptide or a functional portion thereof, a C2c1 polypeptide or a functional portion thereof, a C2c3 polypeptide or a functional portion thereof, and a C2c2 polypeptide or a functional portion thereof.

Embodiment 121: The method according to any one of embodiments 106-120, wherein said cell, tissue, or organ comprises a tissue.

Embodiment 122: The method of embodiment 121, wherein said tissue comprises an epithelium.

Embodiment 123: The method of embodiment 122, wherein said tissue comprise skin.

Embodiment 124: The method of embodiment 121, wherein said tissue comprises an endothelium.

Embodiment 125: The method of embodiment 124, wherein said endothelium comprises a vascular endothelium.

Embodiment 126: The method according to any one of embodiments 106-120, wherein said cell, tissue, or organ comprises an organ.

Embodiment 127: The method of embodiment 126, wherein said organ comprises an organ selected from the group consisting of adrenal gland, appendix, bladder, brain, bronchi, diaphragm, esophagus, gall bladder, heart, hypothalamus, kidneys, large intestine, liver, lungs, lymph nodes, mammary glands, mesentery, ovary, pancreas, pineal gland, parathyroid gland, pituitary gland, prostate, salivary gland, skeletal muscle, small intestine, spinal cord, spleen, stomach, thymus gland, and thyroid.

Embodiment 128: The method according to any one of embodiments 106-120, wherein said cell, tissue, or organ comprises cells.

Embodiment 129: The method of embodiment 128, wherein said cells are selected from the group consisting of invertebrate cells, vertebrate cells, fungal cells, and yeast cells.

Embodiment 130: The method of embodiment 129, wherein said cells comprise mammalian cells.

Embodiment 131: The method of embodiment 130, wherein said cells comprise human cells.

Embodiment 132: The method of embodiment 130, wherein said cells comprise non-human mammalian cells.

Embodiment 133: The method according to any one of embodiments 130-132, wherein said cells comprise lymphocytes, or stem cells.

Embodiment 134: The method of embodiment 133, wherein said cells comprise stem cells selected from the group consisting of adult stem cells, embryonic stem cells, cord blood stem cells and induced pluripotent stem cells.

Embodiment 135: The method according to any one of embodiments 130-132, wherein said cells comprise differentiated somatic cells.

Embodiment 136: The method of embodiment 129, wherein said cells comprise cells from a cell line.

Embodiment 137: The method of embodiment 136, wherein said cells comprise cells from a cell line listed in Table 1.

Embodiment 138: The method of embodiment 136, wherein said cells comprise cells from a cell line selected from the group consisting of HeLa, National Cancer Institute's 60 cancer cell lines (NCI60), ESTDAB database, DU145 (prostate cancer), Lncap (prostate cancer), MCF-7 (breast cancer), MDA-MB-438 (breast cancer), PC3 (prostate cancer), T47D (breast cancer), THP-1 (acute myeloid leukemia), U87 (glioblastoma), SHSY5Y Human neuroblastoma cells, cloned from a myeloma, and Saos-2 cells (bone cancer).

### DEFINITIONS

The term “cargo” as used herein with respect to delivery into a cell, organ, or tissue to any moiety that it is desired to deliver into the cell, organ, or tissue. Illustrative cargos include, but are not limited to organelles, whole chromosomes or bacteria, large nucleic acids or proteins, nucleoprotein complexes, synthetic particles, and the like.

The term “large cargo” refers to cargo ranging in size from about 100 nm, or from about 500 nm, or from about 800 nm, or from about 1 μm, or from about 3 μm, or from about 5 μm up to about 20 μm, or up to about 15 μm, or up to about 10 μm (in length and/or width and/or in diameter). In certain embodiments a large cargo ranges in size from about 100 nm (e.g., DNA and/or RNA in a lipid or liposomal complex) up to about 10 μm (e.g., chromosome, nucleus, etc.).

The term “critical point” refers to the point in a phase diagram at which two phases of a substance initially become indistinguishable from one another. The critical point is the end point of a phase equilibrium curve, defined by a critical pressure Tp and critical temperature Pc. At this point, there is no phase boundary. The most prominent example is the liquid-vapor critical point, the end point of the pressure-temperature curve distinguishing a substance's liquid and vapor. For example, the meniscus between steam and water vanishes at temperatures above 374° C. and pressures above 217.6 atm, forming what is known as a supercritical fluid.

The term “supercritical fluid” or “SCF” refers to a substance at a temperature and pressure above its critical point, where distinct liquid and gas phases do not exist. Close to the critical point, small changes in pressure or temperature can result in large changes in density. The fluid will also be called supercritical even if its temperature is below critical point value as long as the pressure is sufficiently above the critical value.

The term “lightly doped” when used with respect to a substrate indicates that the substrate is doped with ions at a density ranging from about 10¹⁴ to about 10¹⁵ ions/cm³.

The term “heavily doped” when used with respect to a substrate indicates that the substrate is doped with ions at a density of at least about 10¹⁷ ions/cm³.

### BRIEF DESCRIPTION OF THE DRAWINGS

FIG. 1, panels a-b, shows a schematic illustration of a first version of a Laser-actuated Supercritical Injector (LASI) with a second substrate layer present (panel a) and a second substrate layer absent (panel b).

FIG. 2, panels a-b, shows a schematic illustration of a second version of a Laser-actuated Supercritical Injector (LASI) with a second substrate layer present (panel a) and a second substrate layer absent (panel b).

FIG. 3, panels a-b, shows a schematic illustration of a third version of a Laser-actuated Supercritical Injector (LASI) with a second substrate layer present (panel a) and a second substrate layer absent (panel b).

FIG. 4, panels a-e, shows a side view (panel a) and a top view (panel b) of a Laser-actuated Supercritical Injector (LASI). Panels a and b illustrate an LASI comprising a regular “array” of chambers and microchannels. Panels c and d illustrate a top view of staggered array (panel c) and a clustered (panel d) arrangement of chambers and microchannels. Panel e shows one illustrative embodiment where the chambers are disposed at different levels (depths) within the substrate.

FIG. 5, panels a-1, illustrates microfluidic jets with fluorescent beads injected into a hydrogel. Version 1: device schematic (panel a), fluorescent image of injected beads from the top view of the hydrogel (panel b), three-dimensional (3D) reconstructed images of fluorescent beads distribution inside the hydrogel (panel c), single row of injected beads inside the hydrogel (panel d). Version 2: device schematic (panel e), fluorescent image of injected beads from the top view of the hydrogel (panel f), three-dimensional (3D) reconstructed images of fluorescent beads distribution inside the hydrogel (panel g), single row of injected beads inside the hydrogel (panel h). Version 3: device schematic (panel i), fluorescent image of injected beads from the top view of the hydrogel (panel j), three-dimensional (3D) reconstructed images of fluorescent beads distribution inside the hydrogel (panel k), single row of injected beads inside the hydrogel (panel 1).

FIG. 6, panels a-d, illustrate illustrates the use of a laser-assisted supercritical injector (LASI) to introduce a cargo into cells, tissues, or an organ. Panel a) Cargoes are loaded and filled inside the microchannels in the LASI substrate. Panel b) The LASI substrate is applied to cells, tissue, or organ followed by laser scanning of the substrate. Panel c) Laser radiation heats the thin aqueous layer at the surface of the microchannels to the critical point of the fluid contained therein to form a supercritical fluid. Panel d) Photothermal bubbles, induced by the supercritical heating, generate a high speed fluid jet that penetrates the cells, tissue, or organ, and in particular the cell membrane of the cells or the cells comprising the tissue or organ.

FIG. 7, panels a-c, shows schematic illustrations of three illustrative versions of Laser-actuated Supercritical Injector (LASI). Panel a: Version 1. Hemispheric heavily doped silicon cavity. Panel b: Version 2. Silicon cavity with titanium as heating material. Panel c: Version 3. Silicon micro-hole structure.

FIG. 8, panels a-1, shows microfluidic jets with fluorescent beads into hydrogel. Version 1: device schematic (panel a), fluorescent image of injected beads from the top view of the hydrogel (panel b), three-dimensional (3D) reconstructed images of fluorescent beads distribution inside the hydrogel (panel c), single row of injected beads inside the hydrogel (panel d). Version 2: device schematic (panel e), fluorescent image of injected beads from the top view of the hydrogel (panel f), three-dimensional (3D) reconstructed images of fluorescent beads distribution inside the hydrogel (panel g), single row of injected beads inside the hydrogel (panel h). Version 3: device schematic (panel i), fluorescent image of injected beads from the top view of the hydrogel (panel j), three-dimensional (3D) reconstructed images of fluorescent beads distribution inside the hydrogel (panel k), single row of injected beads inside the hydrogel (panel 1).

FIG. 9, panels a-c, Fabrication process of laser induced supercritical injector with heavily doped silicon micro-cavity. Panel a) A layer of 1-μm silicon dioxide (SiO2) was thermally grown. Panel b) The SiO₂ was patterned and etched using reactive ion etching. Panel c) Silicon was etched using SF₆ isotropic etching to create micro-cavities.

FIG. 10, panels a-i, illustrates penetration depth characterized with Agarose hydrogel. Panel a) Green fluorescent beads loaded into the micro-cavities covered by Agarose hydrogel (0.6% w/w). Panel b) Nanosecond pulsed laser automatically scanned across the entire chip. Panel c) Hydrogel with beads injected was inspected using confocal microscope to measure the penetration depth. Penetration depth injected by 80-μm wide micro-cavities: Panel d) top view of fluorescent beads inside the hydrogel. Panel e) three-dimensional image of injected beads, Panel f) single trace of fluorescent beads. Penetration depth injected by 60-μm wide micro-cavities: Panel g) top view of fluorescent beads inside the hydrogel. Panel h) three-dimensional image of injected beads. Panel i) single trace of fluorescent beads. Scale bars: (panel d) 100 μm, (panel g) 100 μm.

FIG. 11, panels a-d, illustrates a fabrication process of in-situ laser induced supercritical injector with metal disk embedded. Panel a) Thermally grown silicon dioxide (SiO₂) was patterned by photo resist (PR) and etched by reactive ion etching. Panel b) 200-nm titanium (Ti) was deposited by electron beam (e-beam) evaporation. Panel c) Silicon was etched by xenon difluoride (XeF₂) isotropic etching. Panel d) Titanium was lifted off by stripping photo resist in Aleg-380.

FIG. 12, panels a-c, shows the penetration depth test by injecting green fluorescent polystyrene beads into the Agarose gel using the in situ laser induced supercritical injector with metal disk. Panel a) Top view of injected fluorescent beads inside the hydrogel. Panel b) Three-dimensional z-stack confocal image of the injected beads trace inside the hydrogel. Panel c) Side view of the confocal image showing the vertical penetration depth. Scale bar: (a) 100 μm.

FIG. 13, panels a-d, illustrate use of a laser-assisted supercritical injector (LASI). Panel a) Cargoes are loaded and filled inside the wells. Panel b) LASI substrate is flipped onto hydrogel, followed by automatic laser scanning Panel c) The thin aqueous layer at the surface of deep holes is heated up to its critical point. Panel d) Photothermal bubbles, induced by the supercritical heating, generate high speed fluid jet to penetrate the hydrogel and cell membrane.

FIG. 14, panels a-e, illustrates one fabrication process and structure of in-situ laser induced supercritical injector (LASI) with silicon deep hole array. Panel a) Fabrication process of the LASI with silicon deep hole array: thermal oxidation of silicon, silicon dioxide (SiO₂) patterning by reactive ion etching (RIE), deep reactive ion etching (DRIE) of silicon. Scanning electron microscope (SEM) images of 5-μm opening, 36-μm deep hole array (panels b, c). SEM images of 3-μm opening, 28-μm deep hole array (panels d, e). Scale bars: (panels b-e) 10 μm.

FIG. 15, panels a-h, illustrates penetration depth characterized with Agarose hydrogel using in-situ laser induced supercritical injector with silicon deep hole array. Penetration depth injected by microhole-array with 5-μm wide opening and 36-μm depth: Panel a) large field of view of beads lateral distribution inside the hydrogel, Panel b) enlarged view of panel a, Panel c) three-dimensional (3D) view of beads inside the hydrogel, (panel d) penetration depth determined by the side view of panel c. Penetration depth injected by microhole-array with 3-μm wide opening and 28-μm depth: Panel e) large field of view of beads lateral distribution inside the hydrogel, Panel f) enlarged view of panel e, Panel g) 3D view of beads inside the hydrogel, Panel h) penetration depth determined by the side view of panel g. Scale bars: (panels a, e) 100 μm, (panels b, 40 μm.

### DETAILED DESCRIPTION

In various embodiments, a Laser-actuated Supercritical Injector (LASI), a device platform that allows high-speed fluidic jet injection into biological samples, such as cells, organs, and tissues (including skin) and methods of use thereof are provided. In certain embodiments the injector devices exploit high-speed fluidic jets that are pushed by bubble explosion in a short period of time. The bubbles are formed when liquid confined in microcavities or holes are heated up to above the supercritical temperature of the fluid. This leads to the formation of a short but ultra-high vapor pressure (e.g., on the order of tens or hundreds of MPa) (supercritical) fluid that ejects the fluid (and any cargo contained therein) out through microchannels. This jet penetrates a cell, organ or tissue juxtaposed to a surface containing the microchannels and the jet provide sufficient force to penetrate into the cell, tissue, or organ leading to effective deliver of a cargo.

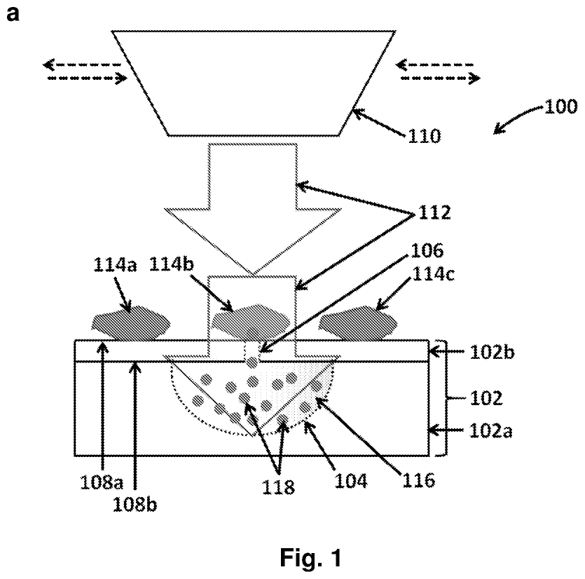

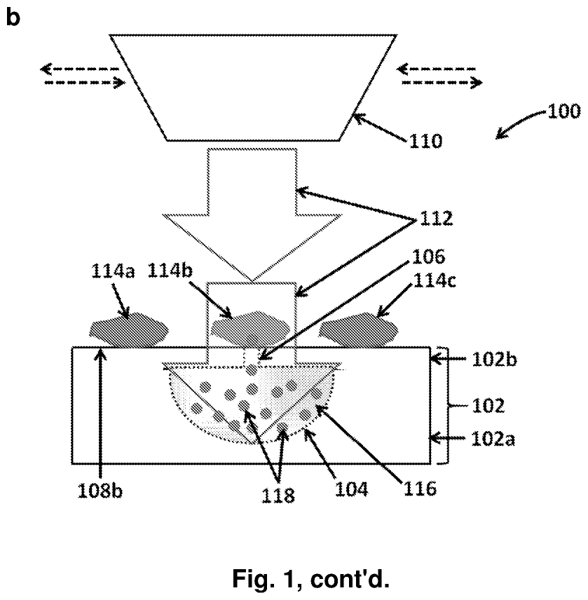

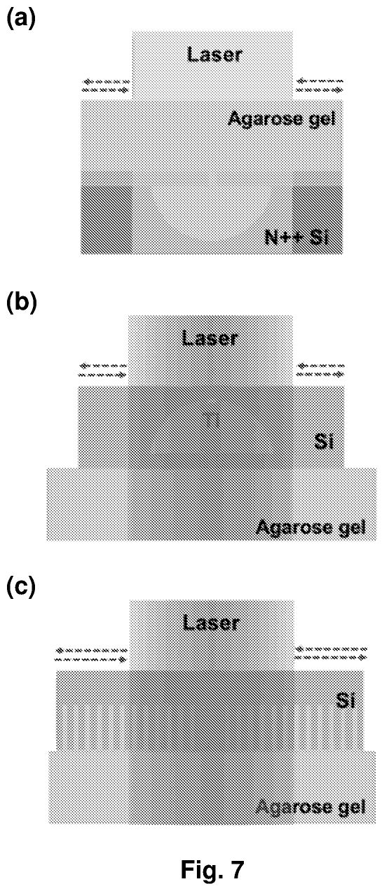

Three illustrative, but non-limiting embodiments of the supercritical injector (see, e.g., FIGS. 1-3, respectively, and FIG. 7, panels a-c, respectively) are described below as well as illustrative uses thereof.

### First Version of a LASI

A first illustrative, but non-limiting version of a laser- actuated supercritical injector (LASI) comprises a substrate that contains a plurality of chambers (cavities) where each chamber is in fluid communication with a microchannel that leads from the chamber and opens to the surface of the substrate. In certain embodiments each chamber is in fluid communication with a single microchannel, while in certain other embodiments, each chamber is in fluid communication with a plurality (e.g., 2, 3, 4, or more) microchannels that each lead from the chamber to the surface of the substrate. When irradiated by a laser, the material forming the chambers is heated by absorption of the laser radiation which heats a fluid contained therein to its supercritical point, where explosive bubbles nucleate and push the fluid medium (and any cargo disposed therein) out through the microchannels forming high speed jets ejecting from the substrate surface. These jets effectively penetrate cells, tissues, or organs disposed adjacent to the surface resulting in delivery of the cargo into the cells, tissues, or organs. In certain embodiments the jets penetrate the cells effectively transfecting the cells, cells comprising the tissue, and/or cells comprising the organ juxtaposed to the surface with the cargo.

This embodiment of a laser- actuated supercritical injector (LASI) is schematically illustrated in FIGS. 1, 4, 5 panel a, and 7 panel a. As illustrated in FIG. 1 the embodiment of the laser-actuated supercritical injector (LASI) 100 shown therein for delivery of a cargo into a cell or tissue comprises a substrate 102 comprising a first layer 102a and optionally comprising a second layer 102b, where the substrate 102 defines an outer surface 108a when the second layer is present (FIG. 1, panel a) or an outer surface 108b when the second layer is absent (FIG. 1, panel b) and where the substrate 102 comprises a plurality of chambers 104 disposed within the substrate 102 where each chamber 104 comprising the plurality of chambers is in fluid communication with one or a plurality of microchannels 106 leading from each chamber to the outer surface of the substrate (108a or 108b) where the microchannel(s) 106 open to the outer surface of the substrate.

Additionally in certain embodiments, all of the chambers are disposed at one level (depth), e.g., in one layer in within the substrate, while in other embodiments, the chambers may be deposited at 2, 3, 4, 5, 6, 7, 8, 9 10, or more different depths (e.g., different layers) in the substrate.

In certain embodiments, the second layer 102b is present. In certain embodiments, layer and at least a portion of the microchannels are disposed in the second layer. In certain embodiments, the second layer comprises an oxide, a nitride, or a polymer. In certain embodiments, the second layer comprises an oxide. In certain embodiments, the second layer comprises SiO₂.

The LASI additionally includes a laser (e.g., a pulse laser) 110 configured to generate laser radiation 112 (light) that illuminates one or more of the chambers 104 comprising the plurality of chambers, where said laser is configured to heat the walls of the illuminated chamber(s) and a fluid 116 contained within the illuminated chamber(s) to transform the fluid into a supercritical fluid that ejects out to the surface (108a in panel a or 108b in panel b) of the substrate 102 through the microchannel(s) 106 opening into the illuminated chamber(s) 104. When cells 114a-114c, tissues, or organs are juxtaposed against the surface the ejected fluid 116 an any cargo 118 contained therein is delivered into (ejected into) the cell(s) 114a-114c, tissue, or organ.

In certain embodiments, the substrate, e.g., the portion of the substrate forming a chamber wall comprises a doped region (e.g., an N doped region or a P doped region). In certain embodiments, the region is lightly doped.

In certain embodiments each chamber comprising the plurality of chambers is in fluid communication with a single microchannel leading from said chamber to the surface (108a in FIG. 1 panel a or 108b in in FIG. 1 panel b) of the substrate 102.

In certain embodiments the second substrate layer 102b is present (see, e.g., FIG. 1, panel a), while in other embodiments the second substrate layer 102b is absent (see, e.g., FIG. 1, panel b).

In certain embodiments, the substrate comprises a material that permits transmission of illumination from said laser to said plurality of chambers to permit heating of the walls of said chambers. In certain embodiments, the substrate comprises a material that provides less than 10% attenuation, or less than 20% attenuation, or less than 30% attenuation, or less than 40% attenuation, or less than 50% attenuation, or less than 60% attenuation, or less than 70% attenuation, or less than 80% attenuation, or less than 90% attenuation, or less than 95% attenuation in said substrate at a depth of 500 μm. In certain embodiments, the substrate comprises silicon or other semiconductor, or other readily machinable material with the desired optical properties (e.g. to pass laser illumination to sufficient depth).

In certain embodiments the substrate comprises silicon. In certain embodiments, the silicon is doped (e.g., P doped or N dopes). In certain embodiments, the substrate is lightly doped silicon.

The configuration of the chambers, microchannels, and substrate composition will be further discussed below.

### Second Version of a LASI

A second illustrative, but non-limiting version of a laser-actuated supercritical injector (LASI) comprises a substrate that contains a plurality of chambers (cavities) where each chamber is in fluid communication with a microchannel that leads from the chamber and opens to the surface of the substrate. In certain embodiments each chamber is in fluid communication with a single microchannel, while in certain other embodiments, each chamber is in fluid communication with a plurality (e.g., 2, 3, 4, or more) microchannels that each lead from the chamber to the surface of the substrate. Each of the chambers contains a “heating element” that comprises a doped region and/or a material (e.g., a metal) that when the chamber(s) are irradiated by a laser, heats up by absorption of the laser radiation which heats a fluid contained in the chamber to its supercritical point, where explosive bubbles nucleate and push the fluid (and any cargo disposed therein) out through the microchannels forming high speed jets ejecting from the substrate surface. These jets effectively penetrate cells, tissues, or organs disposed adjacent to the surface resulting in delivery of the cargo into the cells, tissues, or organs. In certain embodiments the jets penetrate the cells effectively transfecting the cells, cells comprising the tissue, and/or cells comprising the organ juxtaposed to the surface with the cargo.

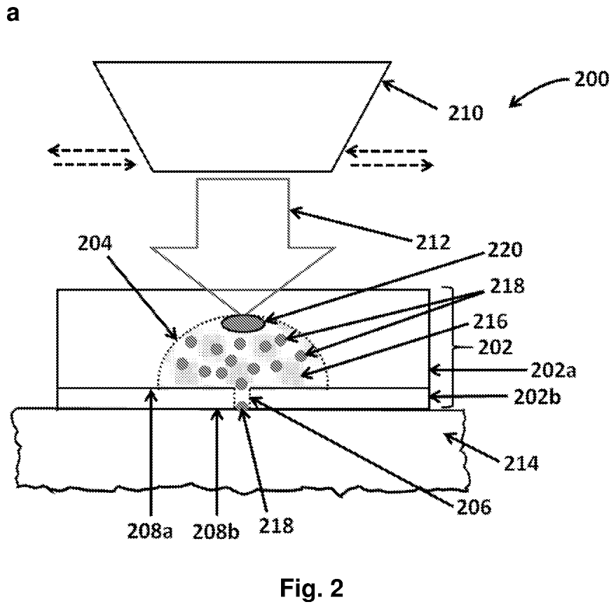

This embodiment of a laser-actuated supercritical injector (LASI) is schematically illustrated in FIGS. 2, 5 panel b, and 7 panel b. As illustrated in FIG. 2 the embodiment of the laser-actuated supercritical injector (LASI) 200 shown therein for delivery of a cargo into a cell or tissue comprises a substrate 202 comprising a first layer 202a, and optionally comprising a second layer 202b, where the substrate 202 defines an outer surface 208a when the second layer is present (FIG. 2, panel a) and 108b when the second layer is absent (FIG. 2, panel b) and where the substrate 202 comprises a plurality of chambers 204 disposed within the substrate 202 where each chamber 204 comprising the plurality of chambers is in fluid communication with one or a plurality of microchannels 206 leading from each chamber to said outer surface of the substrate (208a or 208b) where the microchannel(s) 206 open to the outer surface of the substrate, and where each chamber comprising the plurality of chambers comprises doped region and/or a “heating element” 220 comprising a material that can survive heating to a temperature sufficient to transform a fluid within said chamber to a supercritical fluid when irradiated by a laser. In certain embodiments the “heating element” comprises a metal region or particle disposed within the chamber.

The LASI additionally includes a laser (e.g., a pulse laser) 210 configured to generate laser radiation 212 (light) that illuminates one or more of the chambers 204 comprising the plurality of chambers, where said laser is configured to heat the heating element (e.g. metal region) and a fluid 216 contained within the illuminated chamber(s) to transform the fluid 216 into a supercritical fluid that ejects out to the surface (208a in FIG. 2 panel a or 108b in FIG. 2 panel b) of the substrate 202 through the microchannel(s) 206 opening into the illuminated chamber(s) 204. When cells, tissues 214, or organs are juxtaposed against the surface (108a or 108b) the ejected fluid 216 and any cargo 128 contained therein is delivered into (ejected into) the cell(s), tissue 214, or organ.

In certain embodiments each chamber 204 comprising the plurality of chambers is in fluid communication with a single microchannel 206 leading from the chamber to the surface (208a in FIG. 2 panel a or 208b in FIG. 2 panel b) of the substrate 202. In certain embodiments, all of the chambers are disposed at one level (depth), e.g., in one layer in within the substrate, while in other embodiments, the chambers may be deposited at 2, 3, 4, 5, 6, 7, 8, 9 10, or more different depths (e.g., different layers) in the substrate.

In certain embodiments, the second layer 202b is present. In certain embodiments, layer and at least a portion of the microchannels are disposed in the second layer. In certain embodiments, the second layer comprises an oxide, a nitride, or a polymer. In certain embodiments, the second layer comprises an oxide. In certain embodiments, the second layer comprises SiO₂.

In certain embodiments, the substrate comprises a material that permits transmission of illumination from said laser to said plurality of chambers to permit heating of the walls of said chambers. In certain embodiments, the substrate comprises a material that provides less than 10% attenuation, or less than 20% attenuation, or less than 30% attenuation, or less than 40% attenuation, or less than 50% attenuation, or less than 60% attenuation, or less than 70% attenuation, or less than 80% attenuation, or less than 90% attenuation, or less than 95% attenuation in said substrate at a depth of 500 μm. In certain embodiments, the substrate comprises silicon or other readily machinable material with the desired optical properties (e.g. to pass laser illumination to sufficient depth).

In certain embodiments the substrate comprises silicon. In certain embodiments, the silicon is doped (e.g., P doped or N dopes). In certain embodiments, the substrate comprises lightly doped silicon.

In certain embodiments the second substrate layer 202b is present (see, e.g., FIG. 2, panel a), while in other embodiments the second substrate layer 202b is absent (see, e.g., FIG. 2, panel b).

In certain embodiments the heating element 220 comprises a metal bead deposited within the chamber or a metal region deposited on a surface of the chamber.

In certain embodiments the heating element 220 comprises metal region where the metal region comprises a metal selected from the group consisting of gold, titanium (Ti), TiN, TiCn, TiAlN, tungsten (W), or any other high melting temperature metal or metal alloy. In certain embodiments the metal comprises titanium.

In certain embodiments the heating element 220 (e.g., metal region) comprises a metal disk disposed within the chamber at a wall of the chamber. In certain embodiments the heating element 220 (e.g., metal region) comprises a metal film deposited on the wall of said chamber.

In certain embodiments the metal disk or metal film ranges from about 1 μm up to about 50 μm in average diameter. In certain embodiments the metal disk or metal film comprising the metal region ranges from about 0.05 μm up to about 1 μm in thickness. In certain embodiments the heating element 220 comprises a metal disk having an average diameter of about 3 μm.

### Chambers and Microchannels Comprising LASI Versions 1 and 2

In certain embodiments the chambers comprising the plurality of chambers illustrated in LASI versions 1 and/or 2 are substantially hemispheric. The chambers, however, need not be limited to a hemispheric shape. In various embodiments any of a number of other shapes are suitable. For example, one illustrative alternative comprises teardrop-shaped chambers. In certain embodiments the taper of the tear-drop leads to the microchannel(s). In certain embodiments the chambers are conical-shaped, or cylindrical shaped, or pyramid shaped, or triangular shaped, or substantially spheroid or ovoid. These shapes are illustrative and no-limiting. Essentially any shape that allows the devices to hold the pressure to concentrate fluid jets will generally be acceptable. Using the teachings provided herein, LASI devices comprising chambers having numerous other shapes will be available to one of skill in the art.

In certain embodiments the average volume of the chambers in LASI version 1 and/or LASI version 2 ranges from about 10 fL up to about 100 pL. In certain embodiments the average volume of said chambers is about 1 pL. In certain embodiments the average maximum diameter of the chambers ranges from about 5 μm up to about 100 μm. In certain embodiments the average maximum diameter of the chambers is about 80 μm.

In certain embodiments the microchannels comprising the LASI version 1 and/or LASI version 2 devices range in length from about 0.1 μm up to about 100 μm. In certain embodiments the microchannels have an average length of about 1 μm.

In certain embodiments the microchannels comprising the LASI version 1 and/or LASI version 2 devices range in average diameter from about 100 nm up to about 30 μm. In certain embodiments the microchannels have an average diameter of about 3 μm.

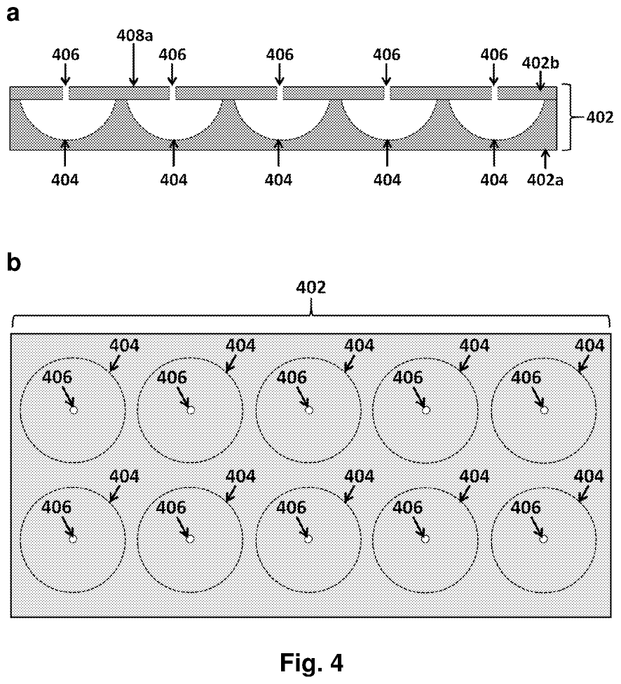

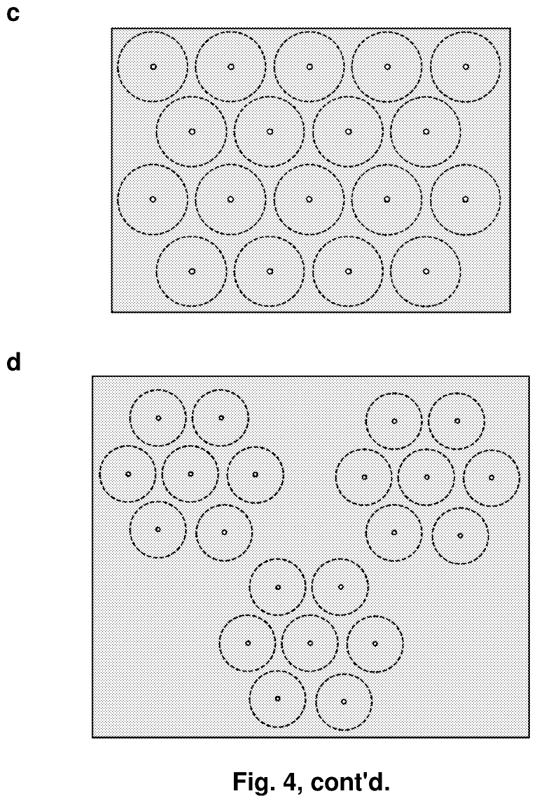

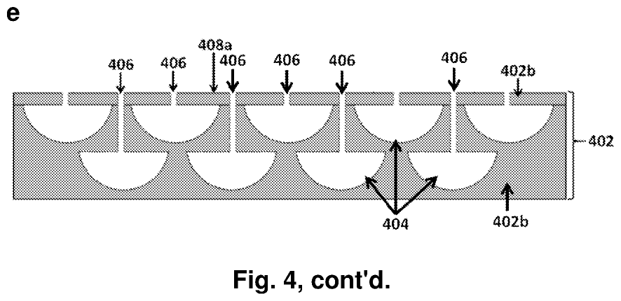

The LASI devices described herein comprise a plurality of microchannels penetrating to the surface of the substrate comprising the device. The density of microchannels (and hence microchannel orifices) is limited by the diameter of the microchannels, or where chambers are present in the device (e.g., LASI version 1 and/or LASI version 2 devices) by the dimensions of the chambers. FIG. 4, panel a, schematically illustrates a side view of one embodiment of a substrate comprising a version 1 LASI device, while panel b illustrates a top view of the same device. As illustrated, panels a and b show a regular “array” of chambers and microchannels. The chambers and microchannels, however, need not be organized in a regular array. For example, a “staggered” array can provide higher density of microchannels and chambers (see, e.g., FIG. 4, panel c). Of course, essentially any desired arrangement can be produced. Thus, for example, FIG. 4, panel d, can provide a clustered (aggregated) arrangement of microchannels and chambers. In certain embodiments such grouping may facilitate loading and delivery of different cargoes, e.g., a different cargo in each group. These patterns are illustrative and non-limiting. Using the teachings provided herein, LASI devices comprising numerous other microchannel and/or microchannel and chamber patterns will be available to one of skill in the art.

In certain embodiments the substrate comprising the LASI version 1 and/or LASI version 2 device comprises at least about 50 microchannels, or at least about 100 microchannels, or at least about 500 microchannels, or at least about 1,000 microchannels, or at least about 2,500 microchannels, or at least about 5,000 microchannels, or at least about 7,500 microchannels, or at least about 10,000 microchannels up to about 4,000,000 microchannels, or up to about 3,000,000 microchannels, or up to about 2,000,000 microchannels, or up to about 1,000,000 microchannels, or up to about 500,000 microchannels, or up to about 250,000 microchannels, or up to about 100,000 microchannels, or up to about 50,000 microchannels. In certain embodiments, the microchannels are present in said substrate at a density of at least about 50 microchannels/cm², or at least about 100 microchannels/cm², or at least about 500 microchannels/cm², or at least about 1,000 microchannels/cm², or at least about 2,500 microchannels/cm², or at least about 5,000 microchannels/cm², or at least about 7,500 microchannels/cm², or at least about 10,000 microchannels/cm², up to about 4,000,000 microchannels/cm², or up to about 3,000,000 microchannels/cm², or up to about 2,000,000 microchannels/cm², or up to about 1,000,000 microchannels/cm², or up to about 500,000 microchannels/cm², or up to about 250,000 microchannels/cm², or up to about 100,000 microchannels/cm², or up to about 50,000 microchannels/cm².

The foregoing configurations of chambers and/or microchannels are illustrative and non-limiting. Using the teaching provided herein, numerous variations of LASI version 1 and/or version 2 substrates will be available to one of skill in the art.

### Third Version of a LASI

A third illustrative, but non-limiting version of a laser-actuated supercritical injector (LASI) comprises a substrate that contains a plurality of microchannels where one end opens to the surface of the substrate while the other end terminates withing the substrate thereby leaving only one opening to each microchannel. In certain embodiments the microchannels (holes) comprise high aspect ratio deep hole arrays with nano- to microscale diameters. The microchannels (holes) are configured to absorb laser energy. When irradiated by a laser, the material forming the microchannels (holes) is heated by absorption of the laser radiation which heats a fluid contained in the channels to its supercritical point, where explosive bubbles nucleate and push the fluid (and any cargo disposed therein) out through the microchannels forming high speed jets ejecting from the substrate surface. These jets effectively penetrate cells, tissues, or organs disposed adjacent to the surface resulting in delivery of the cargo into the cells, tissues, or organs. In certain embodiments the jets penetrate the cells effectively transfecting the cells, cells comprising the tissue, and/or cells comprising the organ juxtaposed to the surface with the cargo.

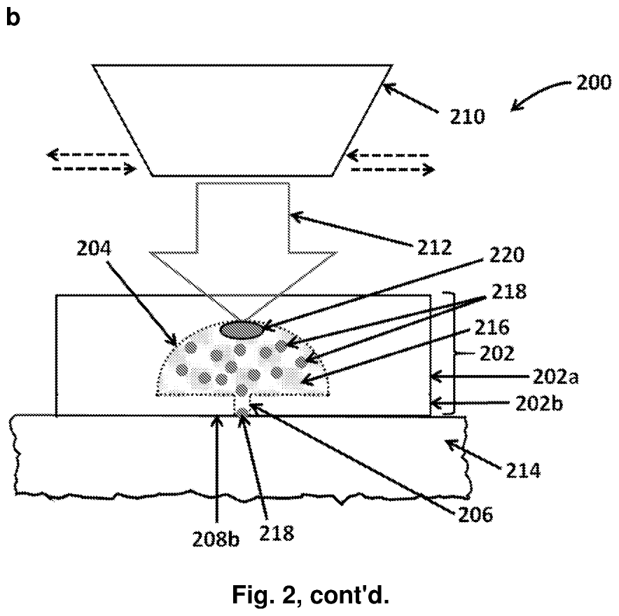

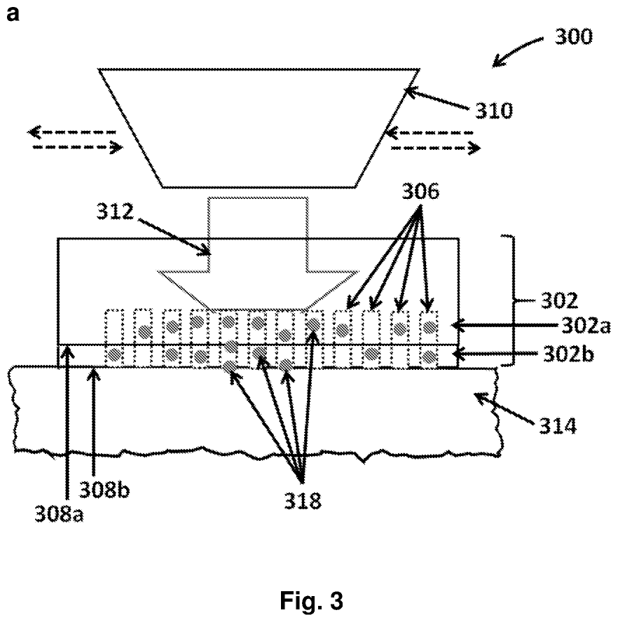

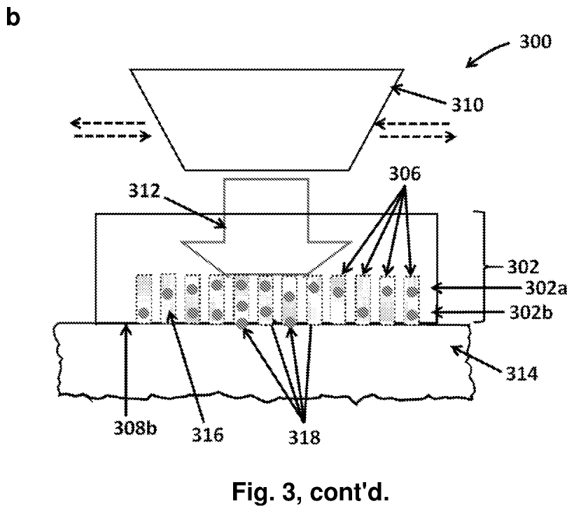

This embodiment of a laser-actuated supercritical injector (LASI) is schematically illustrated in FIGS. 3, 5 panel c, and 7 panel c. As illustrated in FIG. 3 the embodiment of the laser-actuated supercritical injector (LASI) 300 for delivery of a cargo into a cell, tissue 314, or organ, comprises a substrate 302 comprising a first layer 302a, and optionally comprising a second layer 302b, where the substrate 302 defines an outer surface 308a when the second layer is present and 308b when the second layer is absent and comprises a plurality of microchannels 302 (e.g., holes), where each microchannel 306 comprises a first end and a second end, where the first end opens to the outer surface of the substrate (308a when the second layer is present and 308b when the second layer is absent), and the second end of each microchannel is closed, terminating within said substrate 302a. The LASI additionally includes a laser (e.g., a pulse laser) 310 configured to generate laser radiation 312 (light) that the substrate 302 in a region comprising one or more of the microchannels 306, where the laser is configured to heat the walls of the illuminated microchannel(s) and a fluid 316 contained within the illuminated microchannel(s) to transform the fluid into a supercritical fluid that ejects out to the surface (308a in FIG. 3, panel a or 308b in FIG. 3, panel b) of the substrate 302 through the microchannel(s) 306. When cells, tissues 316, or organs are juxtaposed against the surface the ejected fluid 316 an any cargo 318 contained therein is delivered into (ejected into) the cell(s), tissue 316, or organ.

In certain embodiments, the substrate comprises a material that permits transmission of illumination from said laser to said plurality of chambers to permit heating of the walls of said chambers. In certain embodiments, the substrate comprises a material that provides less than 10% attenuation, or less than 20% attenuation, or less than 30% attenuation, or less than 40% attenuation, or less than 50% attenuation, or less than 60% attenuation, or less than 70% attenuation, or less than 80% attenuation, or less than 90% attenuation, or less than 95% attenuation in said substrate at a depth of 500 μm. In certain embodiments, the substrate comprises silicon or other semiconductor, or other readily machinable material with the desired optical properties (e.g. to pass laser illumination to sufficient depth).

In certain embodiments the substrate the substrate 302 comprises a silicon substrate. In certain embodiments the substrate comprises a lightly doped silicon substrate. In certain embodiments the lightly doped silicon substrate comprises an N doped silicon substrate. In certain embodiments, the lightly doped silicon substrate comprises a P doped silicon substrate. In certain embodiments the substrate is doped at a level ranging from about 10¹⁴ to about 10¹⁵ ions/cm³. (Silicon substrates with certain doping range are the current optimal substrates we find. The substrate materials can be broader than silicon.

In certain embodiments, the second layer 302b is present. In certain embodiments, layer and at least a portion of the microchannels are disposed in the second layer. In certain embodiments, the second layer comprises an oxide, a nitride, or a polymer. In certain embodiments, the second layer comprises an oxide. In certain embodiments, the second layer comprises SiO₂. The second layer can act as a transparent mechanical wall to confine the jets.

In certain embodiments the second layer 302b of the substrate is present and comprises an oxide, a nitride, or a polymer and at least a portion of the microchannels 306 are disposed in the second layer. In certain embodiments, the second layer 302b comprises an oxide. In certain embodiments the second layer 302b comprises SiO₂. The SiO₂ layer works as a transparent mechanical wall to confine the jets.

In certain embodiments the microchannels comprising the LASI version 3 device range in length from about 1 μm up to about 500 μm. In certain embodiments the microchannels have an average length of about 28 μm. In certain embodiments the microchannels have an average length of about 36 μm.

In certain embodiments the microchannels comprising the LASI version 3 device have an average diameter that ranges from about 0.1 μm up to about 30 μm. In certain embodiments the microchannels have an average diameter of about 5 μm. In certain embodiments the microchannels have an average diameter of about 3 μm.

In certain embodiments the substrate 302 comprising the LASI version 3 device comprises at least about 50 microchannels, or at least about 100 microchannels, or at least about 500 microchannels, or at least about 1,000 microchannels, or at least about 2,500 microchannels, or at least about 5,000 microchannels, or at least about 7,500 microchannels, or at least about 10,000 microchannels up to about 4,000,000 microchannels, or up to about 3,000,000 microchannels, or up to about 2,000,000 microchannels, or up to about 1,000,000 microchannels, or up to about 500,000 microchannels, or up to about 250,000 microchannels, or up to about 100,000 microchannels, or up to about 50,000 microchannels. In certain embodiments, the microchannels are present in said substrate at a density of at least about 50 microchannels/cm², or at least about 100 microchannels/cm², or at least about 500 microchannels/cm², or at least about 1,000 microchannels/cm², or at least about 2,500 microchannels/cm², or at least about 5,000 microchannels/cm², or at least about 7,500 microchannels/cm², or at least about 10,000 microchannels/cm² up to about 4,000,000 microchannels/cm², or up to about 3,000,000 microchannels/cm², or up to about 2,000,000 microchannels/cm², or up to about 1,000,000 microchannels/cm², or up to about 500,000 microchannels/cm², or up to about 250,000 microchannels/cm², or up to about 100,000 microchannels/cm², or up to about 50,000 microchannels/cm².

### Substrates Utilized in LASI Devices.

The laser-actuated supercritical injectors (LASI) described herein are illustrated using substrates comprising silicon. The substrates need not be so limited. As a general principle it is desirable that the laser energy in the substrate is evenly absorbed across the of the substrate so that maximum size cavitation bubbles are formed on the surface of the microchannels and/or chambers (or the heating element disposed therein). This provides a large fluid jet propelling force.

In general substrate materials are selected to avoid the situation in which light energy gets absorbed within a shallow distance in the substrate which can result in damage to the substrate without triggering sufficient sizes of bubbles since energy is too concentrated. In certain embodiments the materials are selected to provide little to moderate light absorption at a depth on the order of 0.5 to 2 mm, or 0.5 to about 1 mm, or at about 1 mm The absorption will be determined by the wavelength of the laser that is utilized.

Illustrative, but non-limiting, examples of materials other than silicon include, but are not limited to glass substrates with suitable absorption characteristics, or other semiconductors materials such as GaAs or others.

In certain embodiments the substrate comprises a second substrate layer. In certain embodiments the second substrate layer comprises an oxide, a nitride, or a polymer. In certain embodiments, the second layer comprises an oxide. Thus, for example, as illustrated herein, in certain embodiments the second substrate layer can comprise SiO₂. This, however, is illustrative, but non-limiting.

In certain embodiments, second substrate layer, when present, ranges in thickness from about 10 μm up to about 1 mm In certain embodiments, second substrate layer, when present, has an average thickness of about 100 μm.

In various embodiments where the substrate is to be illuminated from “above” (i.e., from the surface through which the microchannels penetrate as illustrated in LASI version 1 (see, e.g., FIG. 1, panel a)), the substrate can have essentially any desirable thickness as long as the substrate is sufficient thick to accommodate the microchannels and/or chambers. However, where the substrate is to be illuminated from “below” (i.e., from the surface opposite the surface through which the microchannels penetrate as illustrated in LASI versions 2 and 3 (see, e.g., FIG. 1, panels b and c))the substrate is desirably thin enough to permit adequate penetration of the illuminating laser radiation. In such instances, the substrate ranges in thickness from about 1 μm up to about 500 μm. In certain embodiments the substrate has an average thickness of about 500 μm. It will be recognized in this regard, that in various embodiments, thickness of the substrate is highly related to the doping in the substrates. Accordingly, in various embodiments, the absorption depth can be from submicron to 500 μm.

In certain embodiments the substrate can be doped. Thus, for example, in the embodiment illustrated by LASI Version 1 the substrate can be heavily doped to enhance absorption of the laser radiation. In certain embodiments such heavy doping ranges from about 10¹⁷ ions/cm³ up to about 10²⁰ ions/cm³. In certain embodiments the heavy doping is about 10²⁰ ions/cm³.

In certain embodiments, as illustrated in the examples, the doped substrate can be an N doped substrate. In certain embodiments the substrate can be a P doped substrate. Illustrative dopants include, but are not limited to P, B, Al, or any other dopants commonly used in semiconductor fabrication.

### Lasers, Optical Systems and Controllers.

The laser-actuated supercritical injector (LASI) devices described herein comprise a laser to heat microchannels and/or chambers in a substrate to generate a supercritical fluid that ejects from microchannels and effectively deliver a cargo into a cell, tissue, or organ. Accordingly, a laser is typically selected to provide laser radiation having a power and wavelength sufficient to heat a fluid within the illuminated chambers and/or microchannels to transform the fluid into a supercritical fluid that ejects out through the microchannel(s) associated with the illuminated region of the substrate.

Accordingly, in certain embodiments, the laser produces illumination at a wavelength ranging from about 380 nm up to about 1100 nm. In certain embodiments the laser produces illumination at a power ranging from about 100 mJ/cm² up to about 1×10⁴ mJ/cm².

In certain embodiments nanosecond pulsed lasers are applied as energy sources for the LASI system. In various embodiments laser radiation in the visible spectrum, such as, for example, a green illumination and can readily be used to pump energy from the top side of a LASI device, as illustrated in FIG. 1, and FIG. 7, panel a. In certain embodiments the laser illumination is at a wavelength of about 532 nm. In certain embodiments the laser (e.g., a green laser) produces illumination at a power of about 200 mJ/cm².

In certain embodiments the laser produces laser radiation in the infrared, e.g., near infrared. Thus, for example, a laser with a wavelength of about 1064 nm, can be used to pump energy from both the top and back sides of a LASI device, as illustrated in FIGS. 2 and 3, and FIG. 7, panels b and c. In certain embodiments the laser produces illumination at a wavelength of about 1064 nm and/or illumination at a power of about 7.6×10³ mJ/cm².

In certain embodiments where the heat absorbing materials are on the top side of the devices, pulsing laser from the top side will yield to a higher energy efficiency, that is, a smaller laser energy is needed to generate photothermal bubbles. However, in a wide range of applications where thick biological samples, e.g., tissue or skin, are targeted, top-side pulsing is not feasible as the laser light can barely penetrate deep into those materials. Therefore, the capability to receive laser energy from the backside while injecting high-speed fluid jets from the top side into the samples has largely broadened the application scope.

In various embodiments the setup of a LASI platform utilizes only a pulsed laser source, a simple optical setup for shaping and collimating a laser beam, and an automatic scanning stage to move the substrate with respect to the laser source. Thus for example, in certain embodiments the LASI substrate is provided on an automatic scanning stage (e.g., an x-y stage).

In certain embodiments the LASI device comprises an optical system that directs the laser beam onto the device substrate. In certain embodiments the automatic scanning stage can be set to move in synchronization with the laser pulsing frequency, to ensure a single laser shot per spot (illumination region on the substrate). In certain embodiments the scanning stage can also enables quick laser pulsing coverage, where the entire LASI device can be scanned within 2 min. In certain embodiments a fluorescence microscope is used to verify cargo injection in two-dimensional(2D) plane. In certain embodiments a confocal microscope can be used to check the cargo injection depth in three-dimensional(3D) images.

In certain embodiments the laser and/or associated optical system, when present, is configured to illuminate a region of the substrate ranging from about 10 μm² up to about 10 cm². In certain embodiments the laser and/or associated optical system, when present, is configured to illuminate a region of the substrate ranging in diameter from about 3 μm up to about 3 cm. In certain embodiments the laser and/or associated optical system, when present, is configured to illuminate a region of the substrate about a 3 mm diameter.

In certain embodiments the LASI comprises a lens system, a mirror system, and/or a mask, and/or a positioning system to directing the laser radiation to a specific region of the substrate. In certain embodiments the injector comprises an objective lens configured to focus optical energy onto the substrate. In certain embodiments the system comprises a collimator and/or a variable aperture, and/or a computer controllable variable aperture where the computer can control and alter in real time the aperture area and/or size.

In certain embodiments the LASI device comprises a controller that controls the laser and/or the position of the LASI substrate with respect to the laser. In certain embodiments the controller adjusts at least one of the pattern of illumination by the laser, the timing of occurrence of light pulses emitted by the laser, the frequency of occurrence of pulses emitted by the laser, the wavelength of pulses emitted by laser, the energy of pulses emitted by the laser, and the aiming or location of pulses emitted by the laser.

In certain embodiments the injector comprises a controller that adjusts the x-y position of said substrate with respect to said laser.

Methods of Injecting a Cargo into a Cell, Tissue, or Organism.

The laser-actuated supercritical injector (LASI) devices described herein can be used to introduce a cargo into cells, tissues or organs with little or no damage to the underlying cellular structure. To demonstrate the fluid injection capability of the three versions of LASI platforms described herein, deionized water filled with fluorescent beads was loaded into cavities (LASI versions 1 and 2) and microchannels (holes) (LASI version 3). A hydrogel was used to mimic the target biological sample(s). The injection patterns in two-dimensional (2D) and penetration depths in three-dimensional (3D) are shown in FIG. 5. To test version 1, a 532 nm laser was pulsed at 14 mJ from the side of hydrogel, which is the top side of the device. Penetration depths of 95 μm into the gel were achieved (see,

FIG. 5, panels a-d). Version 2 was tested upside down by pulsing a 1064 nm laser at 60 mJ from the back side of the silicon substrate to initiate bubbles from the titanium disks in the chambers and achieved 28 μm of penetration deep into the gel (see FIG. 5, panels e-h). Version 3 was tested in the same experimental conditions as version 2 and a 16-μm penetration was achieved (see, FIG. 5, panel i-1).

In view of these results and the results illustrated herein in Example 1, it is demonstrated that the laser-actuated supercritical injector (LASI) devices described herein can readily be used to introduce a cargo into cells, tissues or organs.

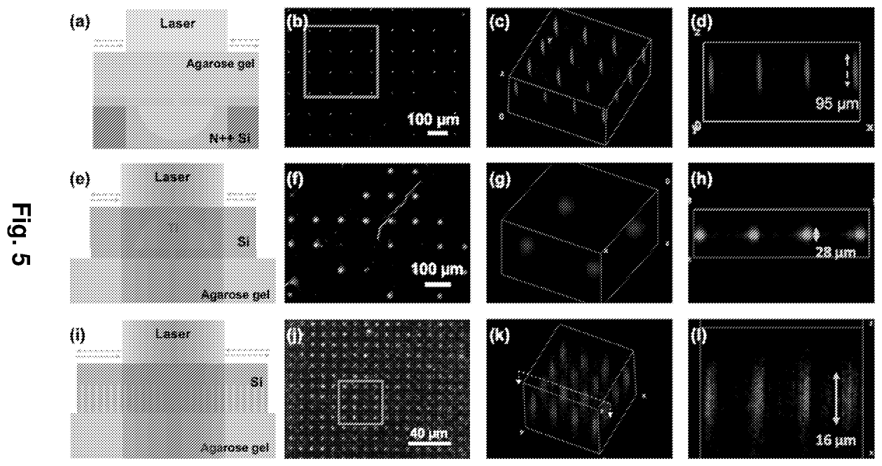

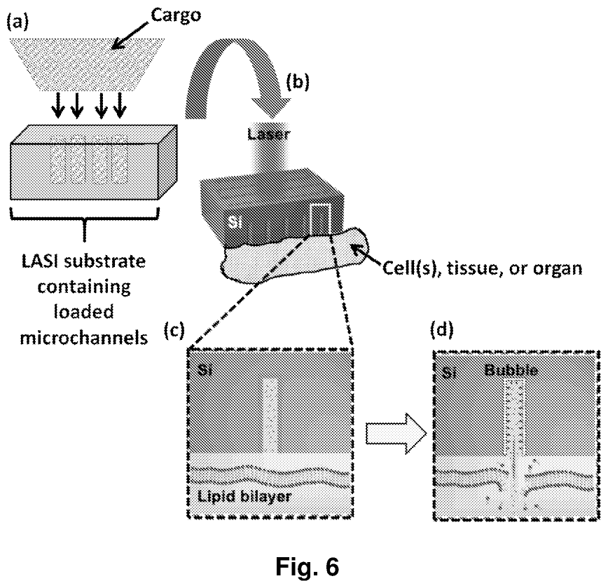

One illustrative, but non-limiting method of using the LASI devices described herein to introduce a cargo into cells, tissue, or organs is illustrated in FIG. 6. As shown in FIG. 6, panel A, a cargo is loaded into the microchannels (or chambers when present) in an LASI substrate. The surface of the LASI substrate showing the microchannel openings is applied to (juxtaposed against) cells, tissue, or organ into which a cargo is to be introduced and the substrate is then irradiated by a laser as illustrated in FIG. 6, panel b. The laser radiation heats the thin aqueous layer at the surface of the microchannels (or chambers when present) to the critical point of the fluid contained therein to form a supercritical fluid. Photothermal bubbles, induced by the supercritical heating, generate a high speed fluid jet that penetrates the cells, tissue, or organ, and in particular the cell membrane of the cells or the cells comprising the tissue or organ illustrated in FIG. 6, panels c and d.

Accordingly, in various embodiments, a method of introducing a cargo into a cell, tissue, or organ, is provided where the method comprises: i) providing a laser-actuated supercritical injector as described herein, where the microchannels and/or chambers when present, are loaded with the cargo in a fluid; juxtaposing said surface of said substrate to a cell, tissue, or organ; and 2) activating a pulse laser to illuminate at least a portion of the LASI substrate to heat the fluid and transform the fluid to a supercritical fluid that ejects out from the microchannels in the LASI substrate and injects into said cell, tissue, or organ thereby delivering the cargo into the cell(s), tissue, or organ. In certain embodiments the cargo is in solution or suspension in a aqueous solution. In certain embodiments the solution or suspension comprises a buffer (e.g., an aqueous buffer, a cell culture medium, etc.). In certain embodiments the cargo comprises a moiety selected from the group consisting of a nucleic acid, a protein, a nucleic acid/protein complex, a carbohydrate, a small organic molecule, an organelle, a nanoparticle, a liposome, a natural chromosome or a natural chromosome fragment, a synthetic chromosome or synthetic chromosome fragment, an intracellular fungus, an intracellular protozoan, DNA and/or RNA packaged in a liposome or a lipid particle, and a vaccine comprising an antigen and an adjuvant.

The forgoing usage is illustrative and non-limiting. Using the teachings provided herein, numerous methods of use of the LASI devices described herein will be available to one of skill in the art.

### Deliverable Materials (Cargo).

It is believed possible to deliver essentially any desired material into a cell using the methods and devices described herein. Such materials include, but are not limited to nucleic acids, proteins, organelles, drug delivery particles, probes, labels, and the like. In embodiments, the cargo comprises one or more moieties selected from the group consisting of comprises a moiety selected from the group consisting of a nucleic acid, a protein, a nucleic acid/protein complex, a carbohydrate, a small organic molecule, an organelle, a nanoparticle, a liposome, a natural chromosome or a natural chromosome fragment, a synthetic chromosome or synthetic chromosome fragment, an intracellular fungus (e.g., Pneumocystis jirovecii, Histoplasma capsulatum, Cryptococcus neoformans, etc.), an intracellular protozoan, (e.g., Apicomplexans (e.g., Plasmodium spp., Toxoplasma gondii, Cryptosporidium parvum, Trypanosomatids (e.g., Leishmania spp., Trypanosoma cruzi, etc.), and the like)DNA and/or RNA packaged in a liposome or a lipid particle, and a vaccine comprising an antigen and an adjuvant.

In certain embodiments the cargo comprises a nucleus, and/or a chloroplast, and/or a nucleolus, and/or a mitochondrion.

In certain embodiments the cargo comprises a whole chromosome, or a chromosome fragment, or a synthetic chromosome (e.g., a BACs (bacterial artificial chromosome)). It is believed the devices and methods described herein can be used to deliver whole or partial natural or synthetic chromosomes. Similar to BACs, large chromosomes or chromosomal fragments that cannot be transduced into most cell types by previous methods can be transferred into cells using the methods described herein, for example, inter alia, to establish models of human trisomy disorders (e.g., Down and Klinefelter syndromes).

In certain embodiments the cargo comprises intracellular pathogens, including but not limited to various bacteria, fungi, and protozoans. The transfection of various inanimate particles is also contemplated. Such particle include, but are not limited to quantum dots, surface-enhanced, Raman scattering (SERS) particles, microbeads, and the like.

In certain embodiments the cargo comprises a moiety selected from the group consisting of a Zinc Finger Nuclease (ZFN), a nucleic acid encoding a ZFN, a Transcription Activator-Like Effector Nuclease (TALEN), a nucleic acid encoding a TALEN, a Clustered Regularly Interspaced Short Palindromic Repeats (CRISPR)-associated protein, and a nucleic acid encoding a CRISPR protein. In certain embodiments the cargo comprises a nucleic acid encoding a CRISPR endonuclease protein and a guide RNA, or a CRISPR endonuclease protein and a guide RNA. In certain embodiments the CRISPR/Cas endonuclease protein comprises a class 2 CRISPR/Cas endonuclease and a guide RNA. In certain embodiments the class 2 CRISPR/Cas endonuclease is a type II CRISPR/Cas endonuclease. In certain embodiments the class 2 CRISPR/Cas endonuclease is a Cas9 polypeptide and the corresponding CRISPR/Cas guide RNA is a Cas9 guide RNA. In certain embodiments the Cas9 protein is selected from the group consisting of a Streptococcus pyogenes Cas9 protein (spCas9) or a functional portion thereof, a Staphylococcus aureus Cas9 protein (saCas9) or a functional portion thereof, a Streptococcus thermophilus Cas9 protein (stCas9) or a functional portion thereof, a Neisseria meningitides Cas9 protein (nmCas9) or a functional portion thereof, and a Treponema denticola Cas9 protein (tdCas9) or a functional portion thereof.

In certain embodiments the cargo comprises a type V or type VI CRISPR/Cas endonuclease or a nucleic acid encoding a type V or type VI CRISPR/Cas endonuclease and, optionally a guide RNA. In certain embodiments the 2 CRISPR/Cas endonuclease is selected from the group consisting of a Cpfl polypeptide or a functional portion thereof, a C2c1 polypeptide or a functional portion thereof, a C2c3 polypeptide or a functional portion thereof, and a C2c2 polypeptide or a functional portion thereof.

It will be recognized that these cargos are intended to be illustrative and non-limiting. Using the teachings provided herein, numerous other cargos, especially large cargos, can be transfected into cells, tissues, and/or organs.

Cell, Tissue, and/or Organs to be Loaded (e.g., Transfected)

In various embodiments the methods and devices described herein can be used to introduce a cargo (e.g., a large cargo) into a cell (or cells), a tissue, and/or an organ. In certain embodiments the cargo is introduced into the interior of a cell, or a cell comprising a tissue or organ thereby effectively transfecting the cell(s) or the cells comprising the tissue or organ with said cargo. In various embodiments the use of the LASI device does not impair the viability of cell(s), tissues, or organs into which the cargo is delivered.

It is believed the methods and devices described herein can be used with essentially any cell having a cell membrane as well as any tissue or organ comprising such cells. Accordingly, in various embodiments, it is contemplated that a cargo can be introduced into essentially any eukaryotic cell, tissue, or organ using the methods and devices described herein. Thus, for example, suitable cells, tissue, or organs that can be cargo-loaded using the the methods described herein include, but are not limited to invertebrate or vertebrate cells, tissues or organs as well as fungal cells and yeast cells. In certain embodiments the cells, tissues, or organs are mammalian, insect, or invertebrate cells, tissues, or organs.

Commonly, the methods described herein will be performed with mammalian cells, tissues, or organs including both human mammalian cells, tissues, or organs and non-human mammalian cells, tissues, or organs (e.g., non-human primate, canine, equine, feline, porcine, bovine, ungulate, rodentia, lagomorph, and the like).

In certain embodiments the cell, tissue, or organ into which a cargo is to be delivered comprises a tissue. In certain embodiments the tissue comprises a tissue selected from the group consisting of muscular tissue, connective tissue, and epithelial tissue. In certain embodiments the tissue comprises skin.

In certain embodiments the cell, tissue, or organ into which a cargo is to be delivered comprises an endothelium (e.g., a vascular endothelium, a lymphatic endothelium, etc.).

In certain embodiments the cell, tissue, or organ into which a cargo is to be delivered comprises an organ or a region of an organ. In certain embodiments the cell, tissue, or organ into which a cargo is to be delivered comprises an organ or a region of an organ selected from the group consisting of adrenal gland, appendix, bladder, brain, bronchi, diaphragm, esophagus, gall bladder, heart, hypothalamus, kidneys, large intestine, liver, lungs, lymph nodes, mammary glands, mesentery, ovary, pancreas, pineal gland, parathyroid gland, pituitary gland, prostate, salivary gland, skeletal muscle, small intestine, spinal cord, spleen, stomach, thymus gland, and thyroid.

In certain embodiments the cell, tissue, or organ into which a cargo is to be delivered comprises a one or more cells. In certain embodiments the cells comprise stem cells or committed progenitor cells. In certain embodiments the stem cells include adult stem cells, fetal stem cells, cord blood stem cells, acid-reverted stem cells, and induced pluripotent stem cells (IPSCs).

In certain embodiments the cells comprise lymphocytes or other differentiated somatic cells.

In certain embodiments the cells comprise cells from a cell line. Suitable cell lines include for example, HeLa, National Cancer Institute's 60 cancer cell lines (NCI60), ESTDAB database, DU145 (prostate cancer), Lncap (prostate cancer), MCF-7 (breast cancer), MDA-MB-438 (breast cancer), PC3 (prostate cancer), T47D (breast cancer), THP-1 (acute myeloid leukemia), U87 (glioblastoma), SHSYSY Human neuroblastoma cells, cloned from a myeloma, Saos-2 cells (bone cancer), and the like.

In certain embodiments suitable cell lines include, but are not limited to cell lines listed in Table 1.

TABLE 1
Illustrative, but non-limiting cells that can be transfected
using the methods described herein.

| Cell line | Organism | Origin tissue |
|---|---|---|
| 293-T | Human | Kidney (embryonic) |
| 3T3 cells | Mouse | Embryonic fibroblast |
| 4T1 | murine | breast |
| 721 | Human | Melanoma |
| 9L | Rat | Glioblastoma |
| A2780 | Human | Ovary |
| A2780ADR | Human | Ovary |
| A2780cis | Human | Ovary |
| A172 | Human | Glioblastoma |
| A20 | Murine | B lymphoma |
| A253 | Human | Head and neck carcinoma |
| A431 | Human | Skin epithelium |
| A-549 | Human | Lung carcinoma |
| ALC | Murine | Bone marrow |
| B16 | Murine | Melanoma |
| B35 | Rat | Neuroblastoma |
| BCP-1 cells | Human | PBMC |
| BEAS-2B | Human | Lung |
| bEnd.3 | Mouse | Brain/cerebral cortex |
| BHK-21 | Hamster | Kidney |
| BR 293 | Human | Breast |
| BxPC3 | Human | Pancreatic adenocarcinoma |
| C2C12 | Mouse | Myoblast cell line |
| C3H-10T1/2 | Mouse | Embryonic mesenchymal |
| | | cell line |
| C6/36 | Asian tiger | Larval tissue |
| | mosquito | |
| C6 | Rat | Glioma |
| Cal-27 | Human | Tongue |
| CGR8 | Mouse | Embryonic Stem Cells |
| CHO | Hamster | Ovary |
| COR-L23 | Human | Lung |
| COR-L23/CPR | Human | Lung |
| COR-L23/5010 | Human | Lung |
| COR-L23/R23 | Human | Lung |
| COS-7 | Monkey | Kidney |
| COV-434 | Human | Ovary |
| CML T1 | Human | CML acute phase |
| CMT | Dog | Mammary gland |
| CT26 | Murine | Colorectal carcinoma |
| D17 | Canine | Osteosarcoma |
| DH82 | Canine | Histiocytosis |
| DU145 | Human | Androgen insensitive carcinoma |
| DuCaP | Human | Metastatic prostate cancer |
| E14Tg2a | Mouse | |
| EL4 | Mouse | |
| EM2 | Human | CML blast crisis |
| EM3 | Human | CML blast crisis |
| EMT6/AR1 | Mouse | Breast |
| EMT6/AR10.0 | Mouse | Breast |
| FM3 | Human | Metastatic lymph node |
| H1299 | Human | Lung |
| H69 | Human | Lung |
| HB54 | Hybridoma | Hybridoma |
| HB55 | Hybridoma | Hybridoma |
| HCA2 | Human | Fibroblast |
| HEK-293 | Human | Kidney (embryonic) |
| HeLa | Human | Cervical cancer |
| Hepa1c1c7 | Mouse | Hepatoma |
| High Five cells | Insect (moth) | Ovary |
| HL-60 | Human | Myeloblast |
| HMEC | Human | |
| HT-29 | Human | Colon epithelium |
| HUVEC | Human | Umbilical vein endothelium |
| Jurkat | Human | T cell leukemia |
| J558L cells | Mouse | Myeloma |
| JY cells | Human | Lymphoblastoid |
| K562 cells | Human | Lymphoblastoid |
| Ku812 | Human | Lymphoblastoid |
| KCL22 | Human | Lymphoblastoid |
| KG1 | Human | Lymphoblastoid |
| KYO1 | Human | Lymphoblastoid |
| LNCap | Human | Prostatic adenocarcinoma |
| Ma-Mel 1, 2, | Human | |
| 3 . . . 48 | | |
| MC-38 | Mouse | |
| MCF-7 | Human | Mammary gland |
| MCF-10A | Human | Mammary gland |
| MDA-MB-231 | Human | Breast |
| MDA-MB-468 | Human | Breast |
| MDA-MB-435 | Human | Breast |
| MDCK II | Dog | Kidney |
| MDCK II | Dog | Kidney |
| MG63 | Human | Bone |
| MOR/0.2R | Human | Lung |
| MONO-MAC 6 | Human | WBC |
| MRC5 | Human (foetal) | Lung |
| MTD-1A | Mouse | |
| MyEnd | Mouse | |
| NCI-H69/CPR | Human | Lung |
| NCI-H69/LX10 | Human | Lung |
| NCI-H69/LX20 | Human | Lung |
| NCI-H69/LX4 | Human | Lung |
| NIH-3T3 | Mouse | Embryo |
| NALM-1 | | Peripheral blood |
| NW-145 | | |
| OPCN/OPCT cell | | |
| lines | | |
| Peer | Human | T cell leukemia |
| PNT-1A/PNT 2 | | |
| Raji | human | B lymphoma |
| RBL cells | Rat | Leukemia |
| RenCa | Mouse | |
| RIN-5F | Mouse | Pancreas |
| RMA/RMAS | Mouse | |
| S2 | Insect | Late stage (20-24 hours old) |
| | | embryos |
| Saos-2 cells | Human | |
| Sf21 | Insect (moth) | Ovary |
| Sf9 | Insect (moth) | Ovary |
| SiHa | Human | Cervical cancer |
| SKBR3 | Human | |
| SKOV-3 | Human | |
| T2 | Human | |
| T-47D | Human | Mammary gland |
| T84 | Human | Colorectal carcinoma/Lung |
| | | metastasis |
| 293-T | Human | Kidney (embryonic) |
| 3T3 cells | Mouse | Embryonic fibroblast |
| 4T1 | murine | breast |
| 721 | Human | Melanoma |
| 9L | Rat | Glioblastoma |
| A2780 | Human | Ovary |
| A2780ADR | Human | Ovary |
| A2780cis | Human | Ovary |
| A172 | Human | Glioblastoma |
| A20 | Murine | B lymphoma |
| A253 | Human | Head and neck carcinoma |
| A431 | Human | Skin epithelium |
| A-549 | Human | Lung carcinoma |
| ALC | Murine | Bone marrow |
| B16 | Murine | Melanoma |
| B35 | Rat | Neuroblastoma |
| BCP-1 cells | Human | PBMC |
| BEAS-2B | Human | Lung |
| bEnd.3 | Mouse | Brain/cerebral cortex |
| BHK-21 | Hamster | Kidney |
| BR 293 | Human | Breast |
| BxPC3 | Human | Pancreatic adenocarcinoma |
| C2C12 | Mouse | Myoblast cell line |
| C3H-10T1/2 | Mouse | Embryonic mesenchymal |
| | | cell line |
| C6/36 | Asian tiger | Larval tissue |
| | mosquito | |
| C6 | Rat | Glioma |
| Cal-27 | Human | Tongue |
| CHO | Hamster | Ovary |
| COR-L23 | Human | Lung |
| COR-L23/CPR | Human | Lung |
| COR-L23/5010 | Human | Lung |
| COR-L23/R23 | Human | Lung |
| COS-7 | Ape | Kidney |
| COV-434 | Human | Ovary |
| CML T1 | Human | CML acute phase |
| CMT | Dog | Mammary gland |
| CT26 | Murine | Colorectal carcinoma |
| D17 | Canine | Osteosarcoma |
| DH82 | Canine | Histiocytosis |
| DU145 | Human | Androgen insensitive carcinoma |
| DuCaP | Human | Metastatic prostate cancer |
| EL4 | Mouse | |
| EM2 | Human | CML blast crisis |
| EM3 | Human | CML blast crisis |
| EMT6/AR1 | Mouse | Breast |
| EMT6/AR10.0 | Mouse | Breast |
| FM3 | Human | Metastatic lymph node |
| H1299 | Human | Lung |
| H69 | Human | Lung |
| HB54 | Hybridoma | Hybridoma |
| HB55 | Hybridoma | Hybridoma |
| HCA2 | Human | Fibroblast |
| HEK-293 | Human | Kidney (embryonic) |
| HeLa | Human | Cervical cancer |
| Hepa1c1c7 | Mouse | Hepatoma |
| High Five cells | Insect (moth) | Ovary |
| HL-60 | Human | Myeloblast |
| HMEC | Human | |
| HT-29 | Human | Colon epithelium |
| HUVEC | Human | Umbilical vein endothelium |
| Jurkat | Human | T cell leukemia |
| J558L cells | Mouse | Myeloma |
| JY cells | Human | Lymphoblastoid |
| K562 cells | Human | Lymphoblastoid |
| Ku812 | Human | Lymphoblastoid |
| KCL22 | Human | Lymphoblastoid |
| KG1 | Human | Lymphoblastoid |
| KYO1 | Human | Lymphoblastoid |
| LNCap | Human | Prostatic adenocarcinoma |
| Ma-Mel 1, 2, | Human | |
| 3 . . . 48 | | |
| MC-38 | Mouse | |
| MCF-7 | Human | Mammary gland |
| MCF-10A | Human | Mammary gland |
| MDA-MB-231 | Human | Breast |
| MDA-MB-468 | Human | Breast |
| MDA-MB-435 | Human | Breast |
| MDCK II | Dog | Kidney |
| MDCK II | Dog | Kidney |
| MG63 | Human | Bone |
| MOR/0.2R | Human | Lung |
| MONO-MAC 6 | Human | WBC |
| MRC5 | Human (foetal) | Lung |
| MTD-1A | Mouse | |
| MyEnd | Mouse | |
| NCI-H69/CPR | Human | Lung |
| NCI-H69/LX10 | Human | Lung |
| NCI-H69/LX20 | Human | Lung |
| NCI-H69/LX4 | Human | Lung |
| NIH-3T3 | Mouse | Embryo |
| NALM-1 | | Peripheral blood |
| NW-145 | | |
| OPCN/OPCT cell | | |
| lines | | |
| Peer | Human | T cell leukemia |
| PNT-1A/PNT 2 | | |
| PTK2 | Rat Kangaroo | kidney |
| Raji | human | B lymphoma |
| RBL cells | Rat | Leukaemia |
| RenCa | Mouse | |
| RIN-5F | Mouse | Pancreas |
| RMA/RMAS | Mouse | |
| Saos-2 cells | Human | |
| Sf21 | Insect (moth) | Ovary |
| Sf9 | Insect (moth) | Ovary |
| SiHa | Human | Cervical cancer |
| SKBR3 | Human | |
| SKOV-3 | Human | |
| T2 | Human | |
| T-47D | Human | Mammary gland |
| T84 | Human | Colorectal carcinoma/Lung |
| | | metastasis |
| THP1 cell line | Human | Monocyte |
| U373 | Human | Glioblastoma-astrocytoma |
| U87 | Human | Glioblastoma-astrocytoma |
| U937 | Human | Leukemic monocytic lymphoma |
| VCaP | Human | Metastatic prostate cancer |
| Vero cells | African green | Kidney epithelium |
| | monkey | |
| WM39 | Human | Skin |
| WT-49 | Human | Lymphoblastoid |
| X63 | Mouse | Melanoma |
| YAC-1 | Mouse | Lymphoma |
| YAR | Human | B cell |

It will be appreciated that the foregoing cell, tissue, or organ types are intended to be illustrative and non-limiting. It will be recognized that numerous other cell, tissue, or organ types can readily be used in the methods and devices described herein.

### Kits.

In certain embodiments , kits are provided for efficient delivery of cargo into cells, tissues, and/or organs. In certain embodiments the kits comprise a container containing a LASI device substrate as described herein. In various embodiments the kits can optionally additionally include any reagents or devices described herein (e.g., reagents, buffers, tubing, indicators, manipulators, etc.) to perform cargo delivery into cells, tissues, and/or organs using the LASI devices described herein.

In addition, the kits optionally include labeling and/or instructional materials providing directions (i.e., protocols) for the use the LASI devices described herein to deliver a cargo into a cell, tissue, and/or organ. In certain embodiments the instruction materials teach methods of loading the LASI substrate(s) with a cargo and can optionally provide recommended laser types and irradiance levels to provide effective delivery of a cargo. In certain embodiments the instruction materials can also include software code for driving a movable stage and a pulse laser to effect delivery of a cargo into target cells, tissues, and/or organs.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Design and Testing of Laser-Assisted Supercritical Injectors

In this example, we demonstrate a large cargo delivery system with direct fluidic injection powered by the large pumping energy from the bubble explosion at supercritical point. Three different device designs are reported to accommodate different application scenarios while sharing the same delivery mechanism (FIG. 7). Instead of using the explosion of bubbles to disrupt cell membranes, we utilize the high-pressure bubble formed by laser irradiation as a pumping source to push out liquid inside a micron-sized cavity or hole structure. Upon laser irradiation, opaque material absorbs the light energy, raises the temperature and heats up surrounding medium to its critical point in just nanoseconds. As thermal expansion of the aqueous medium occurs with no fluidic movement to respond within such a short time, high pressure is built up inside the liquid. As a result, the thin layer of aqueous medium near the heat source turns into a supercritical fluid with huge energy stored, which serves as a pumping source for the high-speed fluidic jet to cut the cell membrane and deliver cargos into the cytosol and nucleus. Our high-speed jet injectors integrate the membrane disruption and active cargo transport into one step for large cargo delivery, free of any excessive needles, particles, or pumping system. The fabrication processes of the devices are designed to be conventional and simple with large-area uniformity. The penetration depth of fluidic jet can be tuned by simply adjusting the etching time in the fabrication process to create different sizes of structures without changing the geometry design. Penetration was demonstrated by injecting 140 nm polystyrene beads into agarose hydrogel which was prepared to have a Young's Modulus similar to mammalian cells. With all the device designs shown in this example, we achieved penetration depths from tens of microns to a hundred microns, indicating the capability of three-dimensional tissue delivery and epidermal in vivo delivery, in addition to intracellular delivery into single layer of cells.

Results and Discussion

Laser induced supercritical injector with heavily doped silicon micro-cavity

Our first LASI platform consists of 10,000 micro-cavities in a 1 cm² heavily doped silicon chip with a 1-μm thick silicon dioxide coating, in which an opening of 3 μm in diameter for each micro-cavity serves as the nozzle for injection (FIG. 7, panel A). Cargoes solution is loaded inside the micro-cavity and samples to be processed are put on top of the device surface. Upon laser irradiation, heavily doped silicon absorbs laser energy and raises its temperature in nanoseconds, which heats the surrounding cargo medium to its critical point and creates cavitation bubbles at the inner surface of the micro-cavity. The rapid expansion of bubbles shoots out the cargo solution through the 3 μm opening into the samples on top. The amount of cargo solution delivered can be tuned by adjusting the micro-cavity size, which determines the volume of liquid pumped out.

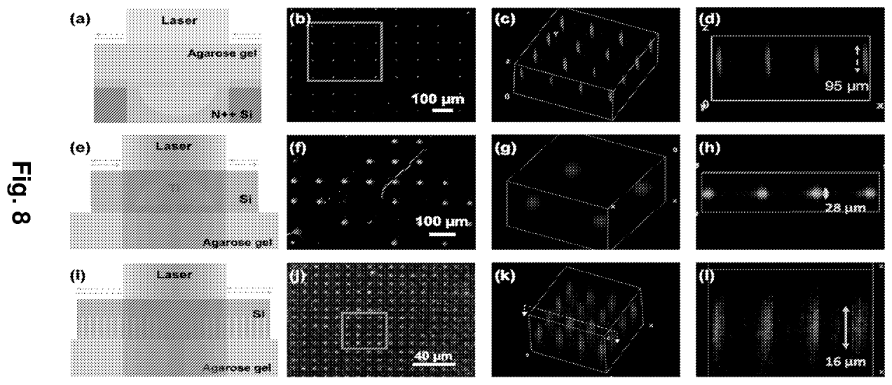

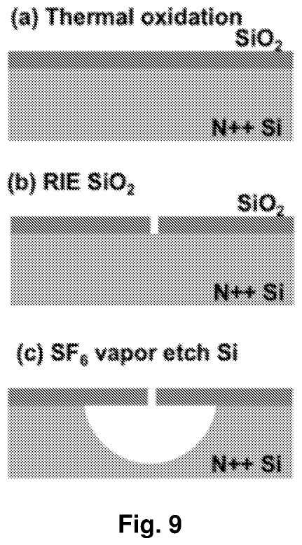

To fabricate the micro-cavity array out of a heavily doped silicon wafer, a 1-μm silicon dioxide was first grown by thermal oxidation, followed by the patterning of the opening array including photolithography and reactive ion etching of silicon dioxide (FIG. 9, panels a, b). A cavity structure was created by etching silicon to wells with a diameter of 80 μm using isotropic vapor etching method (FIG. 9, panel c). The fabricated device was treated with oxygen plasma to improve the wettability of the surface for cargo loading. To prove the filling of cargoes inside micro-cavities, the device was put into a vacuum chamber for a short period after immersion into the cargo solution.

An optical setup was built up to direct the 532-nm nanosecond pulsed laser to the sample located on a translational X-Y stage, which was programmed and controlled by the software, so that the laser beam could scan across the entire chip automatically in 2 min. The programmable stage also enables the activation of the delivery platform at particular locations for potential applications such as drug screening and cell tracking. The laser beam diameter is 3 mm and the laser fluence is 200 mJ/cm2.

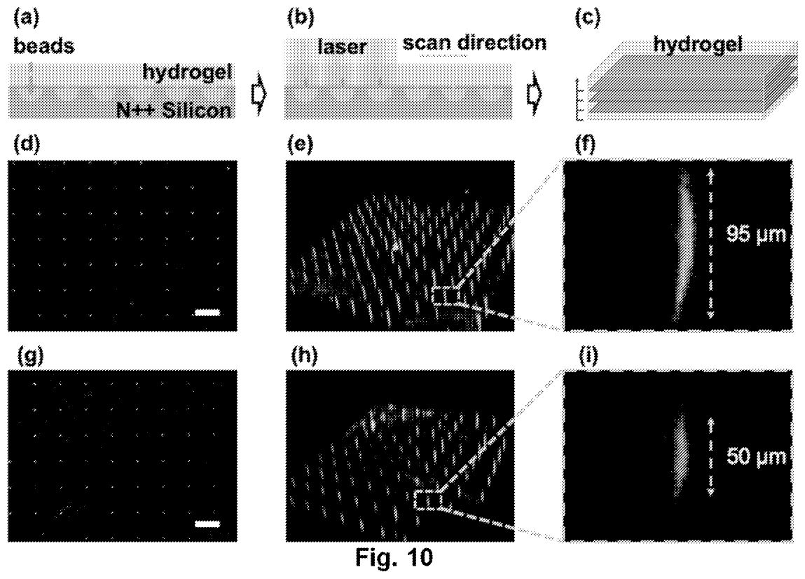

To demonstrate the fluidic jet profile, we tested the device by injecting 140 nm polystyrene fluorescent beads into agarose hydrogel. The agarose was prepared at 0.6% w/w to approximate the Young's Modulus of mammalian cells.^{[37, 38]} After solidification, the hydrogel was cut into 1 cm2 blocks and put on top of the device preloaded with cargoes. The chip and the hydrogel were then transferred to the X-Y stage for laser activation. As cavitation bubbles pushed out the fluidic jet into the hydrogel, fluorescent beads got injected into the hydrogel and remained inside. The hydrogel was then inspected with a confocal microscope, as shown in FIG. 10, panels A-C. Two different sizes of micro-cavities were tested, showing similar injection uniformity in top views (FIG. 10, panels D, E, G, H), indicating its capability of large-area and highly uniform delivery of cargoes. The penetration depths, as expected, were different, as 80-μm wide micro-cavities yielded 95 μm (FIG. 10, panel F) while 60-μm wide ones yielded 50 μm (FIG. 10, panel I).

In Situ Laser Induced Supercritical Injector with Metal Disk Embedded

Despite the promising jet injection capability, our first design would mainly work for thin and transparent biological samples, as the laser light has to travel through the sample before reaching to the silicon surface and may be absorbed. Moreover, it may not be feasible to prepare all the biological samples into a single piece for the laser to fire from one side to the other, restricting the platform from potential in situ delivery application, such as in-vivo epidermal injection. Thus, we developed the second version of LASI device based on the existing design (FIG. 7, panel B).

With a structure similar to the prior device, the second device is provides in situ delivery capability by permitting the laser to fire from the backside of the device instead of from the top side, which frees all the prior requirements imposed on the biological samples. In one illustrative, but non-limiting embodiment, the 532 nm wavelength laser was replaced by a 1064-nm one for less silicon absorption of laser energy. For the same reason, regularly doped silicon wafer was adopted in replacement of the heavily doped silicon. To enhance the optical energy absorption at the cavity surface, a thin layer of titanium was deposited into the cavity and served as the local hot spot to heat up the surrounding aqueous medium.

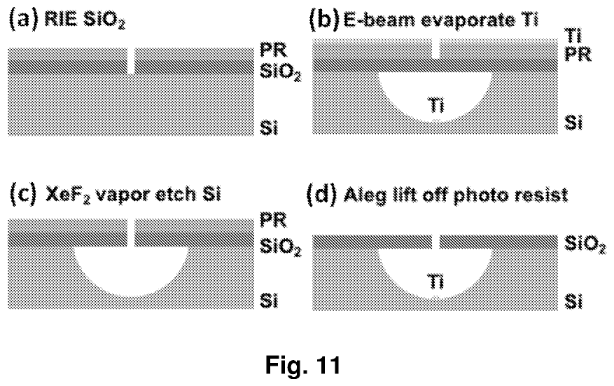

The fabrication process is similar to the prior one, except for the extra step of titanium deposition (FIG. 11). Thermally grown silicon dioxide was patterned and etched to create the 3-μm wide nozzle, followed by isotropic etching of silicon to microwells of 80 μm in diameter. A layer of 100 nm titanium was then deposited by e-beam deposition and titanium on top of the silicon dioxide was lifted off by removing the photoresist in first step.

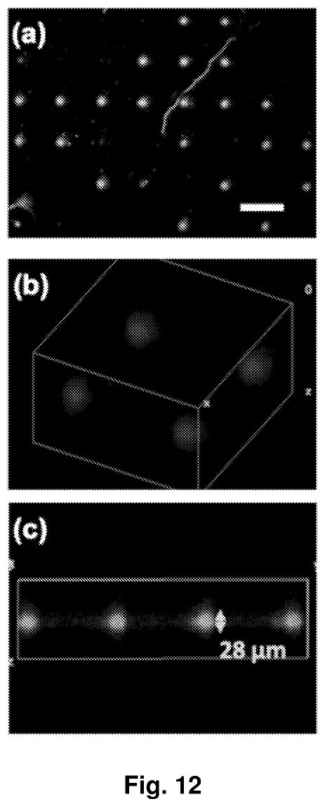

Similarly, we tested the device by injecting 140-nm fluorescent beads into the agarose hydrogel and imaged it with the confocal microscope. The device, preloaded with cargo solution, was flipped over and put onto the hydrogel, followed by the scanning of a 1064 nm laser at 7.6×10 (mJ/cm²). After injection, the device was lifted off and the hydrogel was inspected under confocal microscope. As compared with the beads distribution achieved in prior design, the uniformity was not as good from FIG. 12, as beads at some points of the array were missing. The penetration depth of 28 μm was also shallower. This could be a result of the largely reduced area for light absorption, which, in this example, was titanium disk of 3 μm in diameter compared with the whole inner hemispherical surface of 80 μm in diameter.

In Situ Laser Induced Supercritical Injector with Silicon Deep Hole Array

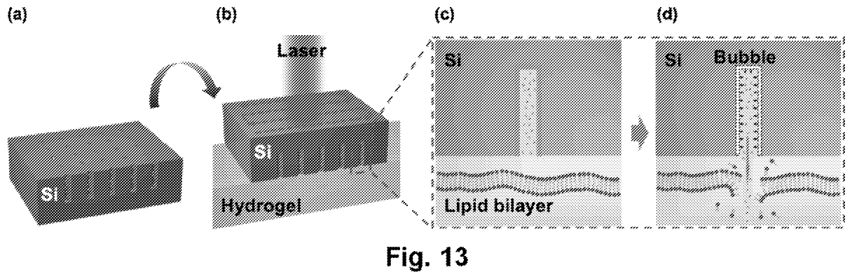

As confirmed by the previous LASI design, cavities with larger diameter yielded deeper penetration into the hydrogel. However, based on the microwell structure, larger cavity size comes with the price of sparser nozzles, as the spacing between two nozzles has to be at least as large as the cavity size. In order to decouple the nozzle density with the cavity volume, we explored a new structure design, taking more advantage of the vertical space under the nozzle, rather than expanding horizontally for larger cavities (FIG. 13). Inherited from the second version of LASI device, the new design kept the in situ delivery capability by still firing laser from the backside of the device and heat up the topside for cavitation bubble generation. As a relatively poor penetration performance was found in the second design using a small 3 μm titanium disk as the light absorbing material, we utilized the silicon itself as a bulk heating component to ensure the sufficient heating surface for large bubble generation.

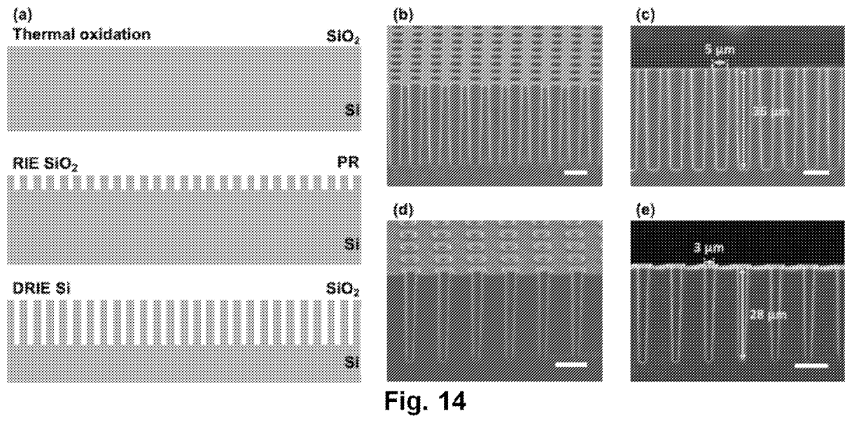

The fabrication process of this design replaces the isotropic etching by directional etching to create high aspect ratio holes (FIG. 14, panel a). Thermally grown silicon dioxide was patterned and etched to create 3-μm opening array, followed by deep reactive ion etching. Two different depths of micro-holes were fabricated, one was 5 μm opening array etched down to 36-μm depth and the other one was 3 μm opening array with 28 μm depth. The scanning electron microscope (SEM) images were taken to provide a thorough view of them (FIG. 14, panels b-e).

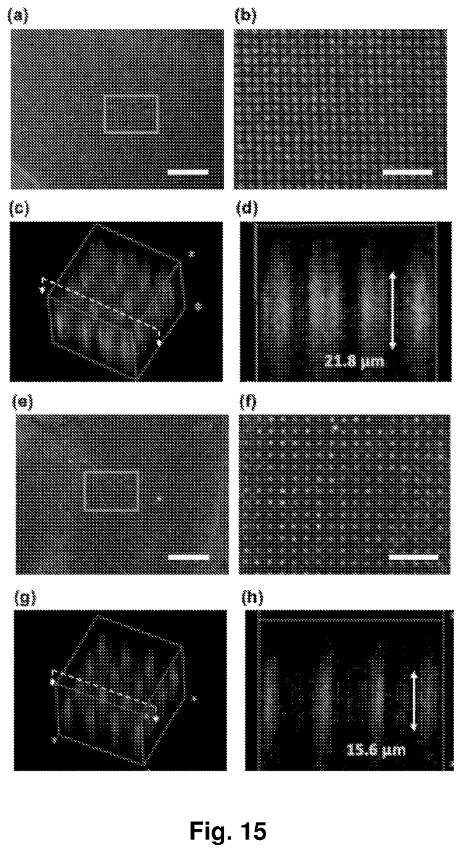

For fair comparison, the new design was tested by injecting the same cargo, 140-nm fluorescent beads, into the same gel using the 1064 nm laser at 7.6×10(mJ/cm2 as the testing done with the second design. The device was flipped over and put onto the hydrogel, followed by laser scanning After laser activation, device was taken off and the hydrogel was inspected using confocal microscope. Compared with the injection results from second design, the uniformity was significantly improved, with large area of clearer and denser beads array injected (FIG. 15, panels a, b, e, f). A penetration depth of 21.8 μm was achieved with an array of 5-μm openings and 36 μm deep holes (FIG. 15, panels c, d), and 15.6 μm was achieved with an array of 3-μm openings and 28-μm deep holes (FIG. 15, panels g, h).

### Conclusion

Large cargo delivery is of great interest in emerging research fields, such as gene editing, metabolic study and intracellular environment probing, due to its capability of delivering into live cells cargoes such as large as whole chromosomes, organelles, and nano devices. Despite the great potential of large cargo delivery in revolutionizing biomedical research, the delivery methods are still limited. Here, we demonstrated a highly efficient delivery method utilizing the initial supercritical pressure of laser-induced cavitation bubbles to inject cargoes into cells without any physical contact with needles or metallic particles.

This reduced the potential of inflammation and uncertainty of drug release, while largely promoting the throughput and accuracy of treatment. We proposed three different versions of designs based on the LASI concept, as one of them works for transparent specimen and the rest two work for in-situ delivery. Injection tests were done using an agarose gel, prepared at a Young's Modulus as similar to cells. Penetration as deep as 95 μm was achieved with highly uniform injection over large area on our first design. We further modified the design for in situ delivery by replacing the laser by 1064 nm laser and the material by regularly doped silicon so as to fire laser from the backside of the device. To decouple the penetration depth with the density of nozzles, we improved the design to take advantage of the vertical space rather than expanding the lateral area. By adopting the third version of design, we managed to inject dense array, with pitch as close as 10 μm, of 140-nm beads into the gel over a large area.

Our prior work conducted large cargo delivery using cavitation bubbles to disrupt cell membranes, followed by mechanical fluidic pumping to transport cargoes, which works for a variety of adherent cells and large cargoes. In this work, we integrated the two processes, membrane disruption and cargo transport into single-step fluidic jet injection at super high speed, which reduced the system complexity and expanded the application scope. The fabrication process was designed to be simple and standard so as to well fit into commercially available processing tools to ensure high yield and uniformity, making it available for people with minimal fabrication experience. The in situ delivery capability enables not only a large variety of cells regardless of their adhesion properties, but also cutting-edge biomedical applications like drug screening and epidermal delivery.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A laser-actuated supercritical injector (LASI) for delivery of a cargo into a cell or tissue, said injector comprising:
a substrate comprising a first layer, and optionally comprising a second layer, where said substrate defines an outer surface and where said substrate comprises a plurality of chambers disposed within the substrate where each chamber comprising said plurality of chambers is in fluid communication with one or a plurality of microchannels leading from each chamber to said outer surface of said substrate where the microchannel(s) opens to the outer surface of said substrate; and
a pulse laser configured to illuminate one or more of the chambers comprising said plurality of chambers, where said laser is configured to heat the walls of the illuminated chamber(s) and a fluid contained with the illuminated chamber(s) to transform said fluid into a supercritical fluid that ejects out to the surface of said substrate through the microchannel(s) opening into the illuminated chamber(s).

2. The laser-actuated supercritical injector of claim 1, wherein said substrate comprises a material that permits transmission of illumination from said laser to said plurality of chambers to permit heating of the walls of said chambers.

3. The laser-actuated supercritical injector of claim 2, wherein said substrate comprises a material that provides less than 10% attenuation, or less than 20% attenuation, or less than 30% attenuation, or less than 40% attenuation, or less than 50% attenuation, or less than 60% attenuation, or less than 70% attenuation, or less than 80% attenuation, or less than 90% attenuation, or less than 95% attenuation in said substrate at a depth of 500 μm.

4. The laser-actuated supercritical injector according of claim 1, wherein said substrate comprises silicon.

5. The laser-actuated supercritical injector according to any one of claims 1-4, wherein said substrate comprises a doped region.

6. The laser-actuated supercritical injector of claim 5, wherein said substrate comprises a lightly doped silicon substrate.

7. The laser-actuated supercritical injector according to any one of claims 5-6, wherein said doped silicon substrate comprises N doped silicon.

8. The laser-actuated supercritical injector according to any one of claims 5-6, wherein said doped silicon substrate comprises P doped silicon.

9. The laser-actuated supercritical injector according to any one of claims 1-8, wherein said substrate is doped at a level ranging from about 10¹³ ions/cm³ up to about 10²⁰ ions/cm³.

10. The laser-actuated supercritical injector according to any one of claims 1-9, wherein each chamber comprising said plurality of chambers is in fluid communication with a single microchannel leading from said chamber to the surface of said substrate.

11. The laser-actuated supercritical injector according to any one of claims 1-9, wherein said substrate comprises said second layer where said second layer and at least a portion of said microchannels are disposed in said second layer.

12. The laser-actuated supercritical injector of claim 11, wherein said second layer comprise a material selected from the group consisting of an oxide, a nitride, or a polymer.

13. The laser-actuated supercritical injector of claim 12, wherein said second layer comprises an oxide.

14. The laser-actuated supercritical injector of claim 13, wherein said oxide comprises SiO₂.

15. The laser-actuated supercritical injector according to any one of claims 1-14, wherein each chamber comprising said plurality of chambers is in fluid communication with one microchannel.

16. The laser-actuated supercritical injector according to any one of claims 1-14, wherein each chamber comprising said plurality of chambers is in fluid communication with a plurality of microchannels.

17. The laser-actuated supercritical injector of claim 15, wherein each chamber comprising said plurality of chambers is in fluid communication with 2, 3, 4, 5, 6, 7, 8, 9, or 10 microchannels.

18. The laser-actuated supercritical injector according to any one of claims 1-17, wherein said plurality of chambers are disposed in a single depth (level) in said substrate.

19. The laser-actuated supercritical injector according to any one of claims 1-17, wherein said plurality of chambers are disposed in two or more depths (levels) in said substrate.

20. A laser-actuated supercritical injector (LASI) for delivery of a cargo into a cell or tissue, said injector comprising:
a substrate comprising a first layer, and optionally comprising a second layer, where said substrate defines an outer surface and where said substrate comprises a plurality of chambers disposed within the substrate where each chamber comprising said plurality of chambers is in fluid communication with one or a plurality of microchannels leading from each chamber to said outer surface of said substrate where the microchannel(s) open to the outer surface of said substrate, and where each chamber comprising said plurality of chambers comprises a doped region and/or a metal region that can survive heating to a temperature sufficient to transform a fluid within said chamber to a supercritical fluid when irradiated by a pulse laser; and
a pulse laser configured to illuminate one or more of the chambers comprising said plurality of chambers, where said laser is configured to heat said metal region(s) in the illuminated chamber(s) and a fluid contained with the illuminated chamber(s) to transform said fluid into a supercritical fluid that ejects out to the surface of said substrate through the microchannel(s) opening into the illuminated chamber(s).

21. The laser-actuated supercritical injector of claim 20, wherein said substrate comprises a material that permits transmission of illumination from said laser to said plurality of chambers to permit heating of the walls of said chambers.

22. The laser-actuated supercritical injector of claim 21, wherein said substrate comprises a material that provides less than 10% attenuation, or less than 20% attenuation, or less than 30% attenuation, or less than 40% attenuation, or less than 50% attenuation, or less than 60% attenuation, or less than 70% attenuation, or less than 80% attenuation, or less than 90% attenuation, or less than 95% attenuation in said substrate at a depth of 500 μm.

23. The laser-actuated supercritical injector of claim 20, wherein said substrate comprises silicon.

24. The laser-actuated supercritical injector according to any one of claims 20-23, wherein each chamber comprising said plurality of chambers is in fluid communication with a single microchannel leading from said chamber to the surface of said substrate.

25. The laser-actuated supercritical injector according to any one of claims 20-23, wherein each chamber comprising said plurality of chambers is in fluid communication with a plurality of microchannels leading from said chamber to the surface of said substrate.

26. The laser-actuated supercritical injector of claim 25, wherein each chamber comprising said plurality of chambers is in fluid communication with 2, 3, 4, 5, 6, 7, 8, 9, or 10 microchannels.

27. The laser-actuated supercritical injector according to any one of claims 20-26, wherein said plurality of chambers are disposed in a single depth (level) in said substrate.

28. The laser-actuated supercritical injector according to any one of claims 20-26, wherein said plurality of chambers are disposed in two or more depths (levels) in said substrate.

29. The laser-actuated supercritical injector according to any one of claims 20-28, wherein said substrate comprises said second layer where said second layer and at least a portion of said microchannels are disposed in said second layer.

30. The laser-actuated supercritical injector of claim 29, wherein said second layer comprise a material selected from the group consisting of an oxide, a nitride, or a polymer.

31. The laser-actuated supercritical injector of claim 30, wherein said second layer comprises an oxide.

32. The laser-actuated supercritical injector of claim 31, wherein said oxide comprises SiO₂.

33. The laser-actuated supercritical injector according to any one of claims 20-32, wherein each chamber comprising said plurality of chambers comprises a doped region.

34. The laser-actuated supercritical injector of claim 33, wherein each chamber comprising said plurality of chambers comprises a heavily doped region.

35. The laser-actuated supercritical injector according to any one of claims 33-34, wherein each chamber comprising said plurality of chambers comprises a P doped region.

36. The laser-actuated supercritical injector according to any one of claims 33-34, wherein each chamber comprising said plurality of chambers comprises an N doped region.

37. The laser-actuated supercritical injector according to any one of claims 20-32, wherein each chamber comprising said plurality of chambers comprises a metal region.

38. The laser-actuated supercritical injector of claim 37, wherein said metal region comprises a metal selected from the group consisting of gold, titanium (Ti), TiN, TiCn, TiAlN, and tungsten (W).

39. The laser-actuated supercritical injector of claim 38, said metal comprises titanium.

40. The laser-actuated supercritical injector according to any one of claims 20-39, wherein said metal region comprises a metal disk disposed within and at a wall of said chamber.

41. The laser-actuated supercritical injector according to any one of claims 20-39, wherein said metal region comprises a metal film deposited on the wall of said chamber.

42. The laser-actuated supercritical injector according to any one of claims 40-41, wherein said metal disk or metal film ranges from about 1 μm up to about 30 μm in average diameter.

43. The laser-actuated supercritical injector according to any one of claims 40-42, wherein said metal disk or metal film comprising said metal region ranges from about 0.05 μm up to about 1 μm in thickness.

44. The laser-actuated supercritical injector according to any one of claims 1-43, wherein the chambers comprising said plurality of chambers are substantially hemispheric.

45. The laser-actuated supercritical injector according to any one of claims 1-43, wherein the chambers comprising said plurality of chambers are substantially cylindrical, or substantially teardrop shaped, or substantially pyramidal shaped, or substantially conical shaped, or substantially triangular shaped.

46. The laser-actuated supercritical injector according to any one of claims 1-45, wherein the average volume of said chambers ranges from about 1 fL up to about 100 pL.

47. The laser-actuated supercritical injector of claim 46, wherein the average volume of said chambers is about 10 pL.

48. The laser-actuated supercritical injector according to any one of claims 1-47, wherein the average maximum diameter of said chambers ranges from about 1 μm up to about 200 μm.

49. The laser-actuated supercritical injector of claim 48, wherein the average maximum diameter of said chambers is about 80 μm.

50. The laser-actuated supercritical injector according to any one of claims 1-49, wherein said microchannels range in length from about 1 μm up to about 500 μm.

51. The laser-actuated supercritical injector of claim 50, wherein said microchannels have an average length of about 1 μm.

52. The laser-actuated supercritical injector according to any one of claims 1-51, wherein said microchannels range in average diameter from about 0.1 μm up to about 30 μm.

53. The laser-actuated supercritical injector of claim 52, wherein said microchannels have an average diameter of about 3 μm.

54. The laser-actuated supercritical injector according to any one of claims 1-53, wherein said substrate comprises at least about 50 microchannels, or at least about 100 microchannels, or at least about 500 microchannels, or at least about 1,000 microchannels, or at least about 2,500 microchannels, or at least about 5,000 microchannels, or at least about 7,500 microchannels, or at least about 10,000 microchannels up to about 4,000,000 microchannels, or up to about 3,000,000 microchannels, or up to about 2,000,000 microchannels, or up to about 1,000,000 microchannels, or up to about 500,000 microchannels, or up to about 250,000 microchannels, or up to about 100,000 microchannels, or up to about 50,000 microchannels.

55. The laser-actuated supercritical injector according to any one of claims 1-54, wherein said microchannels are present in said substrate at a density of at least about 50 microchannels/cm², or at least about 100 microchannels/cm², or at least about 500 microchannels/cm², or at least about 1,000 microchannels/cm², or at least about 2,500 microchannels/cm², or at least about 5,000 microchannels/cm², or at least about 7,500 microchannels/cm², or at least about 10,000 microchannels/cm² up to about 4,000,000 microchannels/cm², or up to about 3,000,000 microchannels/cm², or up to about 2,000,000 microchannels/cm², or up to about 1,000,000 microchannels/cm², or up to about 500,000 microchannels/cm², or up to about 250,000 microchannels/cm², or up to about 100,000 microchannels/cm², or up to about 50,000 microchannels/cm².

56. A laser-actuated supercritical injector (LASI) for delivery of a cargo into a cell, tissue, or organ said injector comprising:
a substrate comprising a first layer, and optionally comprising a second layer, where said substrate defines an outer surface and comprises a plurality of microchannels, where each microchannel comprises a first end and a second end, where the first end opens to the outer surface of said substrate, and the second end of each microchannel is closed, terminating within said substrate; and
a pulse laser configured to illuminate said substrate in a region comprising one or more of the microchannels comprising said plurality of microchannels, where said laser provides laser radiation having a power and wavelength sufficient to heat a fluid within the illuminated microchannels to transform said fluid into a supercritical fluid that ejects out through the illuminated microchannel(s).

57. The laser-actuated supercritical injector of claim 56, wherein said substrate comprises a material that permits transmission of illumination from said laser to said plurality of chambers to permit heating of the walls of said chambers.

58. The laser-actuated supercritical injector of claim 56, wherein said substrate comprises a material that provides less than 10% attenuation, or less than 20% attenuation, or less than 30% attenuation, or less than 40% attenuation, or less than 50% attenuation, or less than 60% attenuation, or less than 70% attenuation, or less than 80% attenuation, or less than 90% attenuation, or less than 95% attenuation in said substrate at a depth of 500 μm.

59. The laser-actuated supercritical injector of claim 56, wherein said substrate comprises silicon.

60. The laser-actuated supercritical injector of claim 56-59, wherein said substrate comprises a doped substrate.

61. The laser-actuated supercritical injector of claim 60, wherein said substrate comprise a lightly doped substrate.

62. The laser-actuated supercritical injector of claim 61, wherein said lightly doped silicon substrate comprises an N doped substrate.

63. The laser-actuated supercritical injector of claim 61, wherein said lightly doped silicon substrate comprises a P doped substrate.

64. The laser-actuated supercritical injector according to any one of claims 56-63, wherein said substrate is doped at a level ranging from about 10¹⁴ to about 10¹⁵ ions/cm³.

65. The laser-actuated supercritical injector according to any one of claims 56-64, wherein said substrate comprises said second layer and at least a portion of said microchannels are disposed in said second layer.

66. The laser-actuated supercritical injector of claim 65, wherein said second layer comprises a material selected from the group consisting of an oxide, a nitride, or a polymer.

67. The laser-actuated supercritical injector of claim 66, wherein said second layer comprises an oxide.

68. The laser-actuated supercritical injector of claim 67, wherein said oxide comprises SiO₂.

69. The laser-actuated supercritical injector according to any one of claims 56-68, wherein said microchannels range in length from about said microchannels range in length from about 1 μm up to about 500 μm.

70. The laser-actuated supercritical injector of claim 69, wherein said microchannels have an average length of about 28 μm.

71. The laser-actuated supercritical injector of claim 69, wherein said microchannels have an average length of about 36 μm.

72. The laser-actuated supercritical injector according to any one of claims 56-70, wherein said microchannels range in average diameter from about 0.1 μm up to about 30 μm.

73. The laser-actuated supercritical injector of claim 72, wherein said microchannels have an average diameter of about 5 μm.

74. The laser-actuated supercritical injector of claim 72, wherein said microchannels have an average diameter of about 3 μm.

75. The laser-actuated supercritical injector according to any one of claims 56-74, wherein said substrate comprises at least about 50 microchannels, or at least about 100 microchannels, or at least about 500 microchannels, or at least about 1,000 microchannels, or at least about 2,500 microchannels, or at least about 5,000 microchannels, or at least about 7,500 microchannels, or at least about 10,000 microchannels up to about 4,000,000 microchannels, or up to about 3,000,000 microchannels, or up to about 2,000,000 microchannels, or up to about 1,000,000 microchannels, or up to about 500,000 microchannels, or up to about 250,000 microchannels, or up to about 100,000 microchannels, or up to about 50,000 microchannels.

76. The laser-actuated supercritical injector according to any one of claims 56-75, wherein said microchannels are present in said substrate at a density of at least about 50 microchannels/cm², or at least about 100 microchannels/cm², or at least about 500 microchannels/cm², or at least about 1,000 microchannels/cm², or at least about 2,500 microchannels/cm², or at least about 5,000 microchannels/cm², or at least about 7,500 microchannels/cm², or at least about 10,000 microchannels/cm² up to about 4,000,000 microchannels/cm², or up to about 3,000,000 microchannels/cm², or up to about 2,000,000 microchannels/cm², or up to about 1,000,000 microchannels/cm², or up to about 500,000 microchannels/cm², or up to about 250,000 microchannels/cm², or up to about 100,000 microchannels/cm², or up to about 50,000 microchannels/cm².

77. The laser-actuated supercritical injector according to any one of claims 1-76, wherein said pulse laser produces illumination at a wavelength ranging from about 380 nm up to about 2000 nm.

78. The laser-actuated supercritical injector of claim 77, wherein said pulse laser produces illumination at a wavelength ranging from about 380 nm up to about 1100 nm.

79. The laser-actuated supercritical injector according to any one of claims 1-78, wherein said pulse laser produces illumination at a power ranging from about 100 mJ/cm² up to about 1×10⁴ mJ/cm².

80. The laser-actuated supercritical injector according to any one of claims 1-79, wherein said pulse laser produces a green illumination.

81. The laser-actuated supercritical injector of claim 80, wherein said laser produces illumination at a wavelength of about 532 nm.

82. The laser-actuated supercritical injector according to any one of claims 80-81, wherein said laser produces illumination at a power of about 200 mJ/cm².

83. The laser-actuated supercritical injector according to any one of claims 1-79, wherein said pulse laser produces an infrared or a near infrared, or a far infrared illumination.

84. The laser-actuated supercritical injector of claim 83, wherein said laser produces illumination at a wavelength of about 1064 nm.

85. The laser-actuated supercritical injector according to any one of claims 83-84, wherein said laser produces illumination at a power of about 7.6×10³ mJ/cm².

86. The laser-actuated supercritical injector according to any one of claims 1-76, wherein said pulse laser is configured to illuminate a region of said substrate ranging from about 1 um² up to about 10 cm².

87. The laser-actuated supercritical injector of claim 86, wherein said pulse laser is configured to illuminate a region of said substrate about a 3 mm diameter.

88. The laser-actuated supercritical injector according to any one of claims 1-87, wherein said injector comprises a lens system, a mirror system, and/or a mask, and/or a positioning system to directing the laser radiation to a specific region of said substrate.

89. The laser-actuated supercritical injector according to any one of claims 1-88, wherein injector comprises an objective lens configured to focus optical energy onto said substrate.

90. The laser-actuated supercritical injector according to any one of claims 1-89, wherein said injector comprises a controller that adjusts at least one of the pattern of illumination by said laser, the timing of occurrence of light pulses emitted by the laser, the frequency of occurrence of pulses emitted by the laser, the wavelength of pulses emitted by the laser, the energy of pulses emitted by the laser, and the aiming or location of pulses emitted by the laser.

91. The laser-actuated supercritical injector according to any one of claims 1-90, wherein said injector comprises a controller that adjusts the x-y position of said substrate with respect to said laser.

92. The laser-actuated supercritical injector according to any one of claims 1-91, wherein said microchannels and/or said chambers when present, are loaded with a cargo.

93. The laser-actuated supercritical injector of claim 92, wherein said cargo is in solution or suspension in a aqueous solution.

94. The laser-actuated supercritical injector of claim 93, wherein said solution or suspension comprises a buffer.

95. The laser-actuated supercritical injector according to any one of claims 92-94, wherein said cargo comprises a moiety selected from the group consisting of a nucleic acid, a protein, a nucleic acid/protein complex, a carbohydrate, a small organic molecule, an organelle, a nanoparticle, a liposome, a natural chromosome or a natural chromosome fragment, a synthetic chromosome or synthetic chromosome fragment, an intracellular fungus, an intracellular protozoan, DNA and/or RNA packaged in a liposome or a lipid particle, and a vaccine comprising an antigen and an adjuvant.

96. The laser-actuated supercritical injector of claim 95, wherein said cargo comprises a cell nucleus, or a mitochondria.

97. The laser-actuated supercritical injector of claim 95, wherein said cargo comprises a nucleic acid encoding an enzyme.

98. The laser-actuated supercritical injector of claim 95, wherein said cargo comprises a moiety selected from the group consisting of a Zinc Finger Nuclease (ZFN), a nucleic acid encoding a ZFN, a Transcription Activator-Like Effector Nuclease (TALEN), a nucleic acid encoding a TALEN, a Clustered Regularly Interspaced Short Palindromic Repeats (CRISPR)-associated protein, and a nucleic acid encoding a CRISPR protein.

99. The laser-actuated supercritical injector of claim 98, wherein said cargo comprises a nucleic acid encoding a CRISPR endonuclease protein and a guide RNA, or a CRISPR endonuclease protein and a guide RNA.

100. The laser-actuated supercritical injector of claim 99, wherein said CRISPR/Cas endonuclease protein comprises a class 2 CRISPR/Cas endonuclease and a guide RNA.

101. The laser-actuated supercritical injector of claim 100, wherein said class 2 CRISPR/Cas endonuclease is a type II CRISPR/Cas endonuclease.

102. The laser-actuated supercritical injector according to any one of claims 100-101, wherein the class 2 CRISPR/Cas endonuclease is a Cas9 polypeptide and the corresponding CRISPR/Cas guide RNA is a Cas9 guide RNA.

103. The laser-actuated supercritical injector of claim 102, wherein said Cas9 protein is selected from the group consisting of a Streptococcus pyogenes Cas9 protein (spCas9) or a functional portion thereof, a Staphylococcus aureus Cas9 protein (saCas9) or a functional portion thereof, a Streptococcus thermophilus Cas9 protein (stCas9) or a functional portion thereof, a Neisseria meningitides Cas9 protein (nmCas9) or a functional portion thereof, and a Treponema denticola Cas9 protein (tdCas9) or a functional portion thereof.

104. The laser-actuated supercritical injector according to any one of claims 100-101, wherein the class 2 CRISPR/Cas endonuclease is a type V or type VI CRISPR/Cas endonuclease.

105. The laser-actuated supercritical injector of claim 104, wherein the class 2 CRISPR/Cas endonuclease is selected from the group consisting of a Cpfl polypeptide or a functional portion thereof, a C2c1 polypeptide or a functional portion thereof, a C2c3 polypeptide or a functional portion thereof, and a C2c2 polypeptide or a functional portion thereof.

106. The laser-actuated supercritical injector according to any one of claims 1-105, wherein a cell tissue, or organ is juxtaposed to said surface of said substrate.

107. A method of introducing a cargo into a cell, tissue, or organ, said method comprising:
providing a laser-actuated supercritical injector according to any one of claims 1-91, wherein said microchannels and/or said chambers when present, are loaded with said cargo in a fluid;
juxtaposing said surface of said substrate to a cell, tissue, or organ; and
activating said pulse laser to illuminate at least a portion of said substrate and to heat said fluid and transform said fluid to a supercritical fluid that ejects out from said microchannels and injects into said cell, tissue, or organ.

108. The method of claim 106, wherein said cargo is in solution or suspension in a aqueous solution.

109. The method of claim 108, wherein said solution or suspension comprises a buffer.

110. The method according to any one of claims 106-109, wherein said cargo comprises a moiety selected from the group consisting of a nucleic acid, a protein, a nucleic acid/protein complex, a carbohydrate, a small organic molecule, an organelle, a nanoparticle, a liposome, a natural chromosome or a natural chromosome fragment, a synthetic chromosome or synthetic chromosome fragment, an intracellular fungus, an intracellular protozoan, DNA and/or RNA packaged in a liposome or a lipid particle, and a vaccine comprising an antigen and an adjuvant.

111. The method of claim 110, wherein said cargo comprises a cell nucleus, or a mitochondria.

112. The method of claim 110, wherein said cargo comprises a nucleic acid encoding an enzyme.

113. The method of claim 110, wherein said cargo comprises a moiety selected from the group consisting of a Zinc Finger Nuclease (ZFN), a nucleic acid encoding a ZFN, a Transcription Activator-Like Effector Nuclease (TALEN), a nucleic acid encoding a TALEN, a Clustered Regularly Interspaced Short Palindromic Repeats (CRISPR)-associated protein, and a nucleic acid encoding a CRISPR protein.

114. The method of claim 113, wherein said cargo comprises a nucleic acid encoding a CRISPR endonuclease protein and a guide RNA, or a CRISPR endonuclease protein and a guide RNA.

115. The method of claim 114, wherein said CRISPR/Cas endonuclease protein comprises a class 2 CRISPR/Cas endonuclease and a guide RNA.

116. The method of claim 115, wherein said class 2 CRISPR/Cas endonuclease is a type II CRISPR/Cas endonuclease.

117. The method according to any one of claims 115-116, wherein the class 2 CRISPR/Cas endonuclease is a Cas9 polypeptide and the corresponding CRISPR/Cas guide RNA is a Cas9 guide RNA.

118. The method of claim 117, wherein said Cas9 protein is selected from the group consisting of a Streptococcus pyogenes Cas9 protein (spCas9) or a functional portion thereof, a Staphylococcus aureus Cas9 protein (saCas9) or a functional portion thereof, a Streptococcus thermophilus Cas9 protein (stCas9) or a functional portion thereof, a Neisseria meningitides Cas9 protein (nmCas9) or a functional portion thereof, and a Treponema denticola Cas9 protein (tdCas9) or a functional portion thereof.

119. The method according to any one of claims 115-116, wherein the class 2 CRISPR/Cas endonuclease is a type V or type VI CRISPR/Cas endonuclease.

120. The method of claim 119, wherein the class 2 CRISPR/Cas endonuclease is selected from the group consisting of a Cpfl polypeptide or a functional portion thereof, a C2c1 polypeptide or a functional portion thereof, a C2c3 polypeptide or a functional portion thereof, and a C2c2 polypeptide or a functional portion thereof.

121. The method according to any one of claims 106-120, wherein said cell, tissue, or organ comprises a tissue.

122. The method of claim 121, wherein said tissue comprises an epithelium.

123. The method of claim 122, wherein said tissue comprise skin.

124. The method of claim 121, wherein said tissue comprises an endothelium.

125. The method of claim 124, wherein said endothelium comprises a vascular endothelium.

126. The method according to any one of claims 106-120, wherein said cell, tissue, or organ comprises an organ.

127. The method of claim 126, wherein said organ comprises an organ selected from the group consisting of adrenal gland, appendix, bladder, brain, bronchi, diaphragm, esophagus, gall bladder, heart, hypothalamus, kidneys, large intestine, liver, lungs, lymph nodes, mammary glands, mesentery, ovary, pancreas, pineal gland, parathyroid gland, pituitary gland, prostate, salivary gland, skeletal muscle, small intestine, spinal cord, spleen, stomach, thymus gland, and thyroid.

128. The method according to any one of claims 106-120, wherein said cell, tissue, or organ comprises cells.

129. The method of claim 128, wherein said cells are selected from the group consisting of invertebrate cells, vertebrate cells, fungal cells, and yeast cells. cells.

130. The method of claim 129, wherein said cells comprise mammalian

131. The method of claim 130, wherein said cells comprise human cells.

132. The method of claim 130, wherein said cells comprise non-human mammalian cells.

133. The method according to any one of claims 130-132, wherein said cells comprise lymphocytes, or stem cells.

134. The method of claim 133, wherein said cells comprise stem cells selected from the group consisting of adult stem cells, embryonic stem cells, cord blood stem cells and induced pluripotent stem cells.

135. The method according to any one of claims 130-132, wherein said cells comprise differentiated somatic cells.

136. The method of claim 129, wherein said cells comprise cells from a cell line.

137. The method of claim 136, wherein said cells comprise cells from a cell line listed in Table 1.

138. The method of claim 136, wherein said cells comprise cells from a cell line selected from the group consisting of HeLa, National Cancer Institute's 60 cancer cell lines (NCI60), ESTDAB database, DU145 (prostate cancer), Lncap (prostate cancer), MCF-7 (breast cancer), MDA-MB-438 (breast cancer), PC3 (prostate cancer), T47D (breast cancer), THP-1 (acute myeloid leukemia), U87 (glioblastoma), SHSYSY Human neuroblastoma cells, cloned from a myeloma, and Saos-2 cells (bone cancer).
